(19) United States
(12) Patent Application Publication
(10) Pub. No.: US 2019/0015335 A1
(43) Pub. Date: Jan. 17, 2019

(54) HEPATOPROTECTIVE PARTICLES AND SYSTEMS AND METHODS OF USE THEREOF

(71) Applicant: South Dakota Board of Regents, Pierre, SD (US)

(72) Inventors: Joshua Reineke, White, SD (US); Mikhail Durymanov, Brookings, SD (US); Anastasia Permyakova, Brookings, SD (US)

(21) Appl. No.: 16/034,156

(22) Filed: Jul. 12, 2018

Related U.S. Application Data
(60) Provisional application No. 62/532,194, filed on Jul. 13, 2017.

(51) Int. Cl. A61K 9/14; A61P 39/00; A61K 31/275; A61K 31/704; A61K 9/51; A61K 31/357; A61K 31/655

(52) U.S. Cl. CPC A61K 9/143; A61P 39/00; A61K 31/275; A61K 31/704; A61K 9/146; A61K 31/655; A61K 9/5115; A61K 9/5123; A61K 9/5153; A61K 31/357; A61K 9/145

(57) ABSTRACT

Compositions for targeted delivery of a protective agent to the liver, and systems and methods for administering the compositions are described. When administered prior to or in combination with one or more chemotherapeutic agents, the compositions containing the protective agent protect the liver without adversely affecting the efficacy of the chemotherapeutic agent(s). Additionally, the use of these compositions enables the administration of higher doses of the chemotherapeutic agent(s). The compositions contain particles, comprising nanoparticles, microparticles, or combinations thereof, which contain a hepatoprotective agent. The systems contain a first formulation comprising particles that contain a protective agent to the liver and a second formulation comprising a chemotherapeutic agent. The particles containing the hapatoprotective agent are short circulating particles.

### CROSS-REFERENCE TO RELATED APPLICATION

This application claims the benefit of and priority to U.S. Application No. 62/532,194 filed Jul. 13, 2017, the contents of which is incorporated by reference in its entirety.

### STATEMENT REGARDING FEDERALLY SPONSORED RESEARCH

This invention was made with Government support under Grant No. HHSN272201000039C awarded by the National Institutes of Health. The Government has certain rights in the invention.

### FIELD OF THE INVENTION

The invention is generally directed to protecting the liver, more particularly to protecting the liver from drug induced-liver injury caused by chemotherapy.

### BACKGROUND OF THE INVENTION

Cytotoxic chemotherapy is a major strategy in a variety of cancer treatments. However, hepatotoxicity of anticancer drugs is one of the concerns associated with chemotherapy. Current chemoprotective agents are designed for systemic, non-targeted applications. These medications also accumulate within the tumor and can exert their chemoprotective effect on tumor tissues to the detriment of chemotherapeutic efficacy.

There is a need for improved compositions and/or systems for protecting the liver, particularly for protecting the liver from drug induced-liver injury caused by chemotherapy.

Therefore, it is an object of the invention to provide improved compositions and/or systems for protecting the liver.

It is a further object of the invention to provide improved chemotherapeutic treatments.

It is a further object of the invention to provide methods for protecting and/or strengthening the liver.

### SUMMARY OF THE INVENTION

Compositions for targeted delivery of protective agents to the liver are disclosed herein. When administered prior to, in combination with, or after the administration of one or more cytotoxic chemotherapeutic agents, these compositions do not detrimentally impact the efficacy of the chemotherapeutic agent(s) while protecting the liver from drug-induced liver injury caused by the chemotherapy. Additionally, the use of these compositions enables the administration of higher doses of the chemotherapeutic agent(s) compared to administration of the same chemotherapeutic agent(s) without these compositions.

Systems and methods for administering the compositions in combination with chemotherapy are also disclosed herein.

Compositions containing particles, typically nanoparticles, microparticles, or combinations thereof, which contain a hepatoprotective agent, may be administered to a subject along with a chemotherapeutic agent for treatment of symptoms of tumor or cancer in a tissue, particularly tissues other than the liver. The particles containing the hepatoprotective agent generally reduce apoptosis of hepatocytes that is otherwise induced when administering the chemotherapeutic agent alone. Further, the particles containing the hepatoprotective agent do not interfere with the efficacy of the chemotherapeutic agent against the tumor or cancer.

### BRIEF DESCRIPTION OF THE DRAWINGS

FIG. 1 is a line graph showing the percentage of released sulforaphane (SFN) from different metal organic frameworks (MOFs), MIL-88A (triangles), MIL-100 (diamonds), and MIL-88B_NH₂ (squares), over time (minutes) in water.

FIG. 2 is a line graph showing the percentage of released SFN from MIL-88A (triangles) and MIL-88B_NH₂ (circles) over time (minutes) in HEPES (4-(2-hydroxyethyl)-1-piperazineethanesulfonic acid)) buffer.

FIG. 3 is a graph of western blot showing the expression levels of phase 2 enzymes (two glutathione-S-transferase (GST) isoforms, GSTP1 and GSTA3) and NAD(P)H:quinone oxidoreductase (NQO1) in AML12 cells following incubation with 10 μM SFN for 12, 24, and 36 hours. The control group was not treated with SFN. Beta-actin was used as a loading control.

FIG. 4 is a bar graph showing the activity of GST (μmol/min per mg of protein) in AML12 cells following incubation of the cells with 10 μM SFN, equivalent dose of SFN encapsulated in MIL-88A (MIL-88A-SFN), or MIL-88A alone for 0, 12, 24, or 48 hours.

FIG. 5 is a bar graph showing the percentage of survived AML12 cells following a 24-hour incubation with (1) control, phosphate buffered saline (PBS); (2) MIL-88A-SFN; (3) 10 μM doxorubicin (DOX); (4) 10 μM DOX after a pretreatment with SFN; (5) 10 μM DOX after a pretreatment with MIL-88A-SFN. The control group was considered to have a 100% cell survival rate. *p<0.05 compared to the control group.

FIG. 6 is a bar graph showing the activities of caspase 3/7 (white bars) and caspase 9 (black bars) per mg of protein (arbitrary unit, a.u.) of AML12 cells following a 24-hour incubation with (1) control, phosphate buffered saline; (2) MIL-88A-SFN; (3) 10 μM doxorubicin (DOX); (4) 10 μM DOX after a pretreatment with SFN; (5) 10 μM DOX after a pretreatment with MIL-88A-SFN. *p<0.05 compared with the control group, Mann-Whitney U test.

FIG. 7 is a bar graph showing the activity of CYP1A1/CYP1B1 (a.u.) of AML12 cells following a 24-hour incubation with (1) control, PBS; (2) MIL-88A-SFN; (3) 10 μM doxorubicin (DOX); (4) 10 μM DOX after a pretreatment with SFN; (5) 10 μM DOX after a pretreatment with MIL-88A-SFN. *p<0.05 compared to the control group.

FIGS. 8A and 8B are graphs of western blot showing the expression levels of GSTA3 and GSTP1 in the liver tissues (FIG. 8A) and in the tumor tissues (FIG. 8B) at day 1 (24 hours) and day 2 (48 hours) upon treatment of the mice bearing B16F1 tumor with SFN or MIL-88A-SFN at a dose of 10 mg SFN per kg of animal, as compared with the control group (treated with PBS) and the group treated with blank MIL-88A.

FIG. 9 is a line graph showing the tumor volume (mm³) in mice over time (days), where the mice were administered with (1) PBS at day 0 and PBS at day 1 (PBS+PBS); (2) PBS at day 0 and DOX at 10 mg per kg of mouse at day 1 (PBS+DOX); (3) SFN at day 0 and DOX at 10 mg per kg of mouse at day 1 (SFN+DOX); (4) MIL-88A-SFN at day 0 and DOX at 10 mg per kg of mouse at day 1 (MIL88A-SFN+DOX). Each group contained eight mice. On the last day, **p<0.001 for the MIL-88A-SFN+DOX and PBS+DOX groups compared to PBS+PBS, *p<0.05 for the SFN+DOX compared to PBS+PBS, one-way ANOVA followed by post-hoc Dunnett's t-test.

FIG. 10 is a bar graph showing the activity of alanine aminotransferase (ALT) in mouse blood serum (mol/min per mL), where the mice were administered with (1) PBS at day 0 and PBS at day 1 (PBS+PBS); (2) PBS at day 0 and DOX at 10 mg per kg of mouse at day 1 (PBS+DOX); (3) SFN at day 0 and DOX at 10 mg per kg of mouse at day 1 (SFN+DOX); or (4) MIL-88A-SFN at day 0 and DOX at 10 mg per kg of mouse at day 1 (MIL88A-SFN+DOX). *p<0.05 compared to the PBS+PBS group.

FIG. 11 is a bar graph showing the activity of caspase 3/7 (a.u.) per μg protein in mice, where the mice were administered with (1) PBS at day 0 and PBS at day 1 (PBS+PBS); (2) PBS at day 0 and DOX at 10 mg per kg of mouse at day 1 (PBS+DOX); (3) SFN at day 0 and DOX at 10 mg per kg of mouse at day 1 (SFN+DOX); or (4) MIL-88A-SFN at day 0 and DOX at 10 mg per kg of mouse at day 1 (MIL88A-SFN+DOX). *p<0.05 compared to the PBS+PBS group.

FIG. 12 is a bar graph showing the activity of CYP1A1/CYP1B1 (a.u.) in mice, where the mice were administered with (1) PBS at day 0 and PBS at day 1 (PBS+PBS); (2) PBS at day 0 and DOX at 10 mg per kg of mouse at day 1 (PBS+DOX); (3) SFN at day 0 and DOX at 10 mg per kg of mouse at day 1 (SFN+DOX); or (4) MIL-88A-SFN at day 0 and DOX at 10 mg per kg of mouse at day 1 (MIL88A-SFN+DOX). *p<0.05 compared to the PBS+PBS group.

FIG. 13 a line graph showing the percentage of released SBN from PLGA particles over time in PBS.

FIG. 14 is a graph of western blot showing the expression levels of NQO1, GSTA3 and GSTP1 in the liver tissues in mice bearing B16F1 tumor one day after the treatment with SBN or PLGA-SBN particles at a dose of 10 mg SBN per kg of mouse, as compared with the control group (treated with PBS) and the group treated with plain PLGA particles. Beta-actin was used as a loading control.

FIG. 15 is a line graph showing tumor volume (mm³) in mice over time (days), where the mice were administered 4 times with time interval of 1 day between cycles with (1) pretreatment with PLGA-SBN particles (corresponds to 10 mg SBN per kg of mouse), followed by DTIC on the next day at a dose of 120 mg per kg of mouse (PLGA-SBN+DTIC); (2) pretreatment with SBN (10 mg per kg of mouse), followed by DTIC on the next day at a dose of 120 mg per kg of mouse (SBN+DTIC); (3) PBS, followed by DTIC on the next day at a dose of 120 mg per kg of mouse (PBS+DTIC); (4) pretreatment with PLGA and free (non-encapsulated) SBN (10 mg SBN per kg of mouse), followed by PBS on the next day (PLGA/SBN+saline); and (5) PBS injected at both days (PBS+saline). The days of treatment with SBN formulations are indicated with open arrows, and the days of treatment with DTIC are shown with closed arrows. On the last day, **p<0.001 for the PLGA-SBN+DTIC, SBN+DTIC and PBS+DTIC groups compared to PBS+saline, *p<0.05 between SBN+DTIC and PBS+DTIC, one-way ANOVA followed by post-hoc Dunnett's t-test.

FIG. 16 is a bar graph showing the activity of alanine aminotransferase (ALT) in mouse serum (IU/L), where the mice were administered 4 times with time interval of 1 day between cycles with (1) PBS at the first day and PBS at the next day (PBS+saline); (2) PBS at the first day and DTIC at 120 mg per kg of mouse at the next day (PBS+DTIC); (3) SBN at the first day and DTIC at 120 mg per kg of mouse at the next day (SBN+DTIC); (4) PLGA-SBN at the first day and DTIC at 120 mg per kg of mouse at the next day (PLGA-SBN+DTIC); or (5) free SBN and plain PLGA particles at the first day and PBS at the next day (PLGA/SBN+saline). *p<0.05 compared to the PBS+saline group.

FIG. 17 is a bar graph showing the bilirubin content in mouse serum (mg/dL), where the mice were administered 4 times with time interval of 1 day between cycles with (1) PBS at the first day and PBS at the next day (PBS+saline); (2) PBS at the first day and DTIC at 120 mg per kg of mouse at the next day (PBS+DTIC); (3) SBN at the first day and DTIC at 120 mg per kg of mouse at the next day (SBN+DTIC); (4) PLGA-SBN at the first day and DTIC at 120 mg per kg of mouse at the next day (PLGA-SBN+DTIC); or (5) free SBN and plain PLGA particles at the first day and PBS at the next day (PLGA/SBN+saline). *p<0.05 compared to the PBS+saline group.

FIG. 18 is a graph of western blot showing the levels of pro-caspase 3 and active caspase 3 in the liver tissues of mice bearing B16F1 tumor, where the mice were administered 4 times with time interval of 1 day between cycles with (1) PBS at the first day and PBS at the next day (PBS+saline); (2) PBS at the first day and DTIC at 120 mg per kg of mouse at the next day (PBS+DTIC); (3) SBN at the first day and DTIC at 120 mg per kg of mouse at the next day (SBN+DTIC); (4) PLGA-SBN at the first day and DTIC at 120 mg per kg of mouse at the next day (PLGA-SBN+DTIC); or (5) free SBN and plain PLGA particles at the first day and PBS at the next day (PLGA/SBN+saline). Beta-actin was used as a loading control.

### DETAILED DESCRIPTION OF THE INVENTION

Compositions for targeted delivery of protective agents to the liver are disclosed herein. Systems and methods for administering these compositions, in combination with chemotherapy, are also disclosed herein. The protective agents, which are delivered to the liver to protect the liver, are generally referred to herein as “hepatoprotective agents”, “hepatoprotective molecules”, or “hepatoprotectants”.

The compositions contain particles in a form suitable for administration to a patient, and particularly for delivery to the liver of the patient. The particles contain a hepatoprotective agent. Optionally, the material that forms the particles includes or is a hepatoprotective agent.

The particles are generally short-circulating or non-stealth particles and do not contain a polymeric surface coating with stealth properties. These particles can be rapidly sequestered by the mononuclear phagocyte system (MPS), especially the liver.

### I. Compositions

The compositions contain particles, typically nanoparticles, microparticles, or combinations thereof. The particles typically contain a hepatoprotective agent. Optionally, the material that forms the particles is or contains a hepatoprotective agent. The particles are generally short-circulating particles. Optionally, the compositions comprises different populations of short-circulating particles and long-circulating particles. The compositions are in a form suitable for administration to a patient. For example, the compositions may be in a suspension form with an aqueous medium or a solid form.

A. Particles

1. Properties of Particles

a. Opsonization, Liver Uptake, and Payload Release

The compositions contain short-circulating or non-stealth particles that are quickly taken up by the liver following administration. Opsonization is the process by which a foreign particle becomes covered with opsonin proteins, thereby making it visible to phagocytic cells. After opsonization, phagocytosis can occur, which is the engulfing and eventual destruction or removal of the foreign particle from the bloodstream. Together these two processes form the main clearance mechanism for the removal of foreign particles larger than the renal threshold limit from the blood. In the case of polymeric particles, which normally cannot be destroyed by the phagocytes, sequestration in the MPS organs, e.g., liver and spleen, typically occurs.

In the bloodstream, opsonins interact with foreign particles by van der Waals, electrostatic, ionic, and hydrophobic/hydrophilic forces. Therefore, the surface features of the particles impact the opsonization process. Hydrophobic and charged particles generally undergo higher opsonization as compared to hydrophilic and neutrally charged particles.

The principle of liver uptake of particles is based on the ability of resident liver macrophages (i.e., Kupffer cells) to recognize and internalize the particles. Digestion of the particles in Kupffer cells/hepatocytes leads to release of the payload of the particles, i.e., hepatoprotective agent, which then reaches adjacent hepatocytes and provides their protective function to the hepatocytes.

b. Properties of Particles

The compositions contain a population of short-circulating or non-stealth particles. The population of short-circulating particles does not include long-circulating or stealth particles.

Long-circulating or stealth particles refer to particles that are invisible to the biological system involved in clearance of foreign particles from the bloodstream. These particles may be composed of or surface-coated with polymers with stealth properties, i.e., high flexibility and high hydrophilicity. In cases of surface coating, the polymeric coatings may cover at least 30%, 40%, 50%, or 60% of the surface of the particles on average. Up to 80%, 85%, 90%, 95%, or even 100% of the surface of the particles may be covered with the coating. Exemplary polymers with stealth properties include natural polysaccharides such as dextran (Dex), polysialic acid (PSA), hyaluronic acid (HA), chitosan (CH), and heparin, as well as synthetic polymers such as polyvinyl pyrrolidone (PVP), polyvinyl alcohol (PVA), polyacrylamide (PAM), poly(N-isopropylacrylamide) (PNIPAM), poly(2-oxazoline), polyethylenimine (PEI), poly(acrylic acid), polymethacrylate, polyelectrolytes, polyhydroxyethylmethacrylate (poly-HEMA), polyalginic acid (alginate), poly(ethylene glycol) (PEG), poly(ethylene glycol) (PEG), polyethylene oxide (PEO), PEG-based copolymers including poloxamers, poloxamines, and polysorbates, and copolymers or blends thereof. Long-circulating particles are slowly taken up by or accumulate in liver tissues and/or liver cells. For example, less than 50%, 40%, 30%, 20%, 10%, or 5% of the long-circulating particles are taken up by or accumulate in liver tissues and/or liver cells after 1, 2, 3, 4, 5, or 6 hours following administration. An exemplary method to detect the uptake of polymeric particles by the liver is described in Bazile, J. Pham. Sci., 1995, 84(4), pp. 493-498.

Short-circulating or non-stealth particles refer to particles that can be rapidly sequestered by the MPS organs, especially the liver. These particles are generally unmodified or “naked” particles that do not contain a polymeric surface coating with natural polysaccharides such as dextran (Dex), polysialic acid (PSA), hyaluronic acid (HA), chitosan (CH), and heparin, or synthetic polymers such as polyvinyl pyrrolidone (PVP), polyvinyl alcohol (PVA), polyacrylamide (PAM), poly(N-isopropylacrylamide) (PNIPAM), poly(2-oxazoline), polyethylenimine (PEI), poly(acrylic acid), polymethacrylate, polyelectrolytes, polyhydroxyethylmethacrylate (poly-HEMA), polyalginic acid (alginate), poly(ethylene glycol) (PEG), poly(ethylene glycol) (PEG), polyethylene oxide (PEO), PEG-based copolymers including poloxamers, poloxamines, and polysorbates, and copolymers or blends thereof. Short-circulating particles are readily taken up by or accumulate in liver tissues and/or liver cells. At least 20% of the short-circulating particles are taken up by or accumulate in liver tissues and/or liver cells within 5 minutes following administration. For example, at least 20%, 25%, 30%, 35%, 40%, 45%, 50%, 60%, 70%, 80%, 90%, or 95% of the short-circulating particles in the composition are taken up by or accumulate in liver tissues and/or liver cells within 5, 10, 15, 20, 30, 45, 60, or 120 minutes following administration.

In some embodiments, the short-circulating particles have hydrophobic surfaces. Particles with a hydrophobic surface can be characterized in bulk as having a water contact angle at or larger than 90°, preferably at or larger than 120°. Such particles possess a preference for the interface or oil-phase of an oil-water interface. The resulting hydrophobic surface lacks affinity for or repels water. In general, the more hydrophobic a particle is, the more that particle tends to not dissolve in, not mix with, or not be wetted by water.

In some embodiments, the long-circulating particles have hydrophilic surfaces. Particles with a hydrophilic surface refers to the property of having affinity for water. Particles with a hydrophobic surface can be characterized in bulk as having a water contact angle smaller than 90°, preferably at or smaller than 30°. Such particles are primarily soluble in aqueous solutions and/or have a tendency to absorb water. In general, the more hydrophilic a particle is, the more that particle tends to dissolve in, mix with, or be wetted by water.

Hydrophobicity of a particle can be quantified by measuring its partition coefficient between water (or a buffered aqueous solution) and a water-immiscible organic solvent, such as octanol, methylene chloride, or methyl tert-butyl ether. If after equilibration a greater concentration of the particle is attained in the organic solvent than in water, the particle is considered hydrophobic. Alternatively, if after equilibration a greater concentration of the particle is attained in water than in the organic solvent, the particle is considered hydrophilic. For example, if the organic solvent is octanol, then a positive log P value indicates that the particle is hydrophobic and a negative log P value indicates that the particle is hydrophilic.

2. Metal Organic Frameworks (MOFs)

The short-circulating particles may be or contain metal organic frameworks (MOFs). MOFs are inorganic-organic hybrid coordination polymers that have repeating units of inorganic moieties connected via organic moieties resulting in one-, two- or three-dimensional networks. They are also commonly referred to as hybrid porous solids or porous coordination polymers (PCP). The inorganic moieties, also denoted as secondary building units (SBU), can be represented by different examples, such as simple metal ions, clusters, chains (1D), layers (2D), or even inorganic 3D arrangements. The SBUs of MOFs are periodically connected via spacer organic molecules (e.g., organic polydentate ligands) that are also denoted as spacer ligands, linking ligands, linkers, organic linkers, or organic ligands. These organic linkers may themselves incorporate metal atoms (as in porphyrin). Some MOFs have unsaturated metal sites on the walls of the pores, and these sites are available to bind drug molecules.

An exemplary group of MOFs for delivering hepatoprotective agents or other active agents for protection of the liver is the MIL (Materials of Institut Lavoisier) family. The MIL family of MOFs generally contains divalent, trivalent, or tetravalent metal centers and carboxylic acid bridging ligands. Exemplary MOFs from the MIL family are MIL-47, MIL-53, MIL-69, MIL-88A, MIL-88B, MIL-88Bt, MIL-88C, MIL-88D, MIL-89, MIL-100, MIL-101, MIL-102, and MIL-127.

The MOFs generally have large pore sizes, e.g., between about 5 and 100 Å, between about 20 and 50 Å, between about 10 and 20 Å, or between about 25 and 35 Å. The MOFs also have large surface areas in general, e.g., between about 500 and 8,000 m²/g, between about 2,500 and 7,000 m²/g, or between about 3,000 and 6,000 m²/g. Preferred MOFs have a size range of 50-1,000 nm and an aspect ratio below 4. Aspect ratio for a population of particles or a sample thereof can be determined by characterizing a population of the particles via electron microscopy techniques, e.g., SEM and TEM. The aspect ratio can be calculated as the average ratio of the highest to the lowest dimension for the population of particles or the sample thereof. Suitable aspect ratios for the MOFs typically range from 1 to 5, optionally, from 1 to 2.

Any suitable MOF may be used in the particles. Exemplary MOFs and methods for making MOFs are described in U.S. patent application publication Nos. 2010/0209354 and 2010/0286022.

The particles may be formed from a porous MOF depicted using a three-dimensional succession of units corresponding to a general Formula (I) below:

MₘOₖXₗLₚSₛ Formula (I)

in which:

M represents a metal ion;

m is an integer greater than or equal to one (m≥1), for example, 1, 2, 3, 4, 5, 6, 7, 8, 9, 10, 11, or 12;

O is oxygen atom;

k is an integer greater than or equal to zero (k≥0), for example, 0, 1, 2, 3, 4, 5, 6, 7, or 8;

X is a counter ion, and can be for example, OH⁻, Cl⁻, F⁻, I⁻, Br⁻, SO₄²⁻, NO₃⁻, ClO₄⁻, PF₆⁻, BF₃⁻, R¹—(COO)ₙ⁻, R—(SO₃)ₙ⁻, R—(PO₃)ₙ⁻, in which R¹ is a hydrogen atom, an optionally substituted linear or branched C₁ to C₁₂ alkyl, and n=1, 2, 3, or 4;

l is an integer greater than or equal to zero (l≥0), for example, 0, 1, 2, 3, 4, 5, 6, 7, 8, 9, 10, 11, 12, 13, 14, 15, 16, 17, or 18;

L is a spacer ligand, also referred to as a linking ligand, a linker, or an organic ligand;

p is an integer greater than or equal to one (p≥1), for example, 1, 2, 3, 4, 5, or 6;

S is molecule of solvent and can be for example, H₂O, EtOH, MeOH, and

s is an integer greater than or equal to zero (l≥0), for example, 0, 1, 2, 3, 4, 5, or 6.

The metal ions of MOFs are often coordinate to labile solvent molecules (S) or counter ions (X), which can be removed after activation of the framework (upon heating). In solution, typically counter ion and/or solvent molecules are included in the structure of a MOF. For example, heating MIL-100 at 150° C. results in the removal of coordinated H₂O and counter ion X, leading to the formation of unsaturated metal sites. Contacting dried MOF with humid air or water generally saturates these sites again. In preferred MOFs, X is present and 1 is an integer between 1 and 5, for example, 1, 2, 3, 4, or 5.

a. Metal Ions of Secondary Building Units

The metal organic frameworks include secondary building units (SBUs) containing one, two, three, or more metal ions. As shown in Formula I, the MOF may contain 1 to 12 metal ions in general. Optionally, the MOF-structure can contain different metal ions, and is commonly referred to as mixed-metal MOFs.

Examples of metal ions that form SBUs include iron (Fe³⁺, Fe²⁺, or both), zinc, and aluminum. Other metal ions include Na⁺, K⁺, Mg²⁺, and Ca²⁺, and combinations thereof. Alternative metal ions may also include Li⁺, Rb⁺, Sr²⁺, Ba²⁺, Sc³⁺, Ti⁴⁺, Zr⁴⁺, Ta³⁺, Cr³⁺, Mo³⁺, W³⁺, Mn³⁺, Ru³⁺, Ru²⁺, Os³⁺, Os^{2+,} Co³⁺, Co²⁺, Ni²⁺, Ni⁺, Pd²⁺, Pd⁺, Pt²⁺, Pt⁺, Cu²⁺, Cu⁺, Au⁺, Ga³⁺, In^{3+,} Si⁴⁺, Si²⁺, Ge⁴⁺, Ge²⁺, Sn⁴⁺, Sn²⁺, Bi⁵⁺, Bi³⁺, Mn²⁺, and Cr³⁺. Additional metal ions, including Tb, Dy, Eu, Gd, and Yb, may also form SBUs in MOFs, particularly for their optical properties.

In some forms, the particles are or contain iron-based MOFs, comprising iron ions with an oxidation state of +3 or +2 in a octahedral coordination geometry with a coordination number of 6. When M in Formula (I) represents iron, Fe, each occurrence of Fe can be identical or different, i.e., independently representing the metal ion Fe³⁺ or Fe²⁺. Such particles may be nanoparticles, optionally, which are administered to a patient for protection of the liver.

Coordination number refers to the number of bonds for which the two electrons shared in the bond originate from the same atom. The coordination number can vary from 2 to 16. A complexed metal ion having a specific coordination number can have one or more possible coordination geometries. For example, complexed iron ions having a coordination number of six may have a hexagonal planar geometry, a trigonal prism geometry, or an octahedral geometry. For example, MIL-88A, MIL-100, MIL-101, and MIL-53 contain Fe³⁺ with an octahedral coordination geometry, organized in oxo-centered trimers of iron octahedra in the cases of MIL-88A, MIL-100 and MIL-101 or chains of corner-sharing iron octahedra in the case of MIL-53.

In some forms, particles are or contain zinc-based MOFs. Zinc-based MOF materials are described in U.S. Published Application No. 2003/0078311 to Muller, et al. U.S. Published Application No. 2003/0004364 to Yaghi, et al. describes zinc-based isoreticular metal-organic framework (IRMOF) materials. IRMOF materials refer to MOF materials based on frameworks of the same topology. The particles may be nanoparticles, optionally, which are administered to a patient for protection of the liver.

If desired, robust, stable MOFs can be formed with hard acid and hard base, based on the hard and soft acid and base theory as described in Pearson, R. G., J. Am. Chem. Soc., 85, 3533 (1963). When the carboxylate group is a hard Lewis base, biocompatible hard Lewis acids, such as Fe³⁺, can be used in the construction of robust MOFs.

b. Linkers/Spacer Ligands

In MOFs, “spacer ligand” or “linker” refers to a ligand coordinating to at least two metal ions. Ligands can be neutral molecules or can have an overall negative charge (anionic). Linkers participate in providing distance between these metal ions, resulting in one-, two- or three-dimensional network of repeating organic and inorganic units. Linkers can be organic materials that also provide biocompatibility and increase loading capacity of the particles with the protective agent. Generally, the linkers contain one or more functional groups such as carboxylates, imidazolates, tetrazolates, pyrazolates, amines, hydroxys, and/or phosphonates. Exemplary linkers include anionic O (e.g., polycarboxylates, polyphosphonates, polyhydroxys) and N (e.g., imidazolates, polypyrazolates, and polytetrazolates) donors.

Exemplary linkers include a radical R bearing one or more (e.g., up to six) carboxylate groups, which may be monodentate or bidentate, i.e., possibly including one or two points of attachment to the metal:

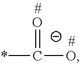

where * denotes the point in the linkers where the carboxylate attaches to the radical R; and # denotes the possible points of attachment of the carboxylate to the metal ion.

Specifically, the radical R in the linkers (or spacer ligands) represents:

(i) a C₁₋₁₂ alkyl, C₂₋₁₂ alkene or C₂₋₁₂ alkyne radical;

(ii) a fused or non-fused monocyclic or polycyclic aryl radical, comprising 6 to 50 carbon atoms;

(iii) a fused or non-fused monocyclic or polycyclic heteroaryl radical, comprising 1 to 50 carbon atoms; or

(iv) an organic radical comprising a metal element chosen from the group comprising ferrocene, porphyrin, phthalocyanin and a Schiff base R^{X1}R^{X2}—C═N—R^{X3},

in which R^{X1} and R^{X2} are independently a hydrogen atom, a linear, branched or cyclic, optionally substituted C₁₋₁₂ alkyl, C₂₋₁₂ alkene or C₂₋₁₂ alkyne radical, or an optionally branched and/or substituted monocyclic or polycyclic aryl radical comprising 6 to 50 carbon atoms;

and R^{X3} is a linear, branched or cyclic, optionally substituted C₁₋₁₂ alkyl, C₂₋₁₂ alkene or C₂₋₁₂ alkyne radical, or an optionally branched and/or substituted monocyclic or polycyclic aryl radical comprising 6 to 50 carbon atoms; and the radical R is optionally substituted with one or more groups,

where each group is independently a C₁₋₁₀ alkyl; C₂₋₁₀ alkene; C₂₋₁₀ alkyne; C₃₋₁₀ cyclo-alkyl; C₁₋₁₀ heteroalkyl; C₆₋₁₀ aryl; C₃₋₁₀ heteroaryl; C₅₋₂₀ heterocycle; C₁₋₁₀ alkyl C₆₋₁₀aryl; C₁₋₁₀ alkyl C₃₋₁₀ heteroaryl; C₁₋₁₀ alkoxy; C₆₋₁₀ aryloxy; C₃₋₁₀ heteroalkoxy; C₃₋₁₀ heteroaryloxy; C₁₋₁₀ alkylthio; C₆₋₁₀ arylthio; C₁₋₁₀ heteroalkylthio; C₃₋₁₀-heteroarylthio; F; Cl; Br; I; —NO₂; —CN; —CF₃; —CH₂CF₃; —CHCl₂; —OH; —CH₂OH; —CH₂CH₂OH; —NH₂; —CH₂NH₂; —NHCOH; —COOH; —CONH₂; —SO₃H; —CH₂SO₂CH₃; —PO₃H₂; —B(OR^{G1})₂; or a function -GR^{G1} in which G is —O—, —S—, —NR^{G2}C(═O)—, —S(═O)—; —SO₂—; —C(═O)O—, —C(═O)NR^{G2}—, OC(═O)—, —NR^{G2}C(═O)—, —OC(═O)O—, —OC(═O)NR^{G2}—, —NR^{G2}C(═O)O—, —NR^{G2}C(═O)NR^{G2}, C(═S)—, —C(═S)S—, —SC(═S)—, —SC(═S)S—, C(═NR^{G2})—, —C(═NR^{G2})O—, —C(═NR^{G2})NR^{G3}, —OC(═NR^{G2}), NR^{G2}C(═NR^{G3})—, —NR^{G2}SO₂—, —C(═NR^{G2})NR^{G3}, —OC(═NR^{G2}), NR^{G2}C(═NR^{G3})—, —NR^{G2}SO₂—, —NR^{G2}SO₂NR^{G3}, —NR^{G2}C(═S)—SC(═S)NR^{G2}—, NR^{G2}C(═S)S—, —NR^{G2}C(═S)NR^{G2}—, —SC(═NR^{G2})—, —C(═S)NR^{G2}—, —OC(═S)NR^{G2}—, —NR^{G2}C(═S)O—, —SC(═O)NR^{G2} NR^{G2}C(═O)S—, —C(═O)S—, —SC(═O)—, —SC(═O)S—, —C(═S)O—, OC(═S)—, —OC(═S)O— or —SO₂NR^{G2};

where each occurrence of R^{G1}, R^{G2} and R^{G3} is, independently, a hydrogen atom; a halogen atom; or a linear, branched or cyclic, optionally substituted C₁₋₁₂ alkyl, C₁₋₁₂ heteroalkyl, C₂₋₁₀ alkene or C₂₋₁₀ alkyne group; or a C₆₋₁₀ aryl, C₃₋₁₀ heteroaryl, C₅₋₁₀ heterocycle, C₁₋₁₀ alkyl C₆₋₁₀ aryl or C₁₋₁₀ alkyl C₃₋₁₀ heteroaryl group in which the aryl, heteroaryl or heterocyclic radical is optionally substituted; and, when G represents —NR^{G2}—, R^{G1} and R^{G2} together with the nitrogen atom to which they are attached form an optionally substituted heterocycle or heteroaryl.

Examples of linking ligands useful in forming the MOF particles include fumaric acid, trimesic acid, and terephthalic acid, and derivatives thereof, typically in a deprotonated form (or in the form of a carboxylate anion) suitable for coordination to the metal ions. Other linking ligands include, for example, citric acid, malic acid, tartaric acid, methanoic acid, ethanoic acid, propanoic acid, butanoic acid, valeric acid, caproic acid, caprylic acid, capric acid, lauric acid, mylistic acid, palmitic acid, stearic acid, oleic acid, linoleic acid, linolenic acid, cyclohexanecarboxylic acid, phenylacetic acid, benzoic acid, oxalic acid, malonic acid, succinic acid, glutaric acid, gallic acid, adipic acid, pimelic acid, suberic acid, maleic acid, phthalic acid, isophthalic acid, hemimellitic acid, trimellitic acid, succinic anhydride, maleic anhydride, phthalic anhydride, glycolic acid, lactic acid, hydroxybutyric acid, mandelic acid, 3,3′,5,5′-azobenzene tetracarboxylic acid, glyceric acid, malic acid, zoledronic acid, tartaric acid, citric acid, and ascorbic acid, and derivatives thereof, typically in a deprotonated form.

Additional exemplary linking ligands include imidazole, 1-methylimidazole, 2-methylimidazole, and 4-methylimidazole, as well as derivatives thereof, typically in a deprotonated form (or in the form of an imidazolate anion) suitable for coordination to the metal ions.

Additional exemplary linking ligands also include γ-cyclodextrin and its derivatives.

Optionally, the linking ligands have hepatoprotective activity. The incorporation of organic ligands that have hepatoprotective activity into MOFs allows for controlled release of hepatoprotective molecules as a function of the rate of degradation of the MOF particles. Thus, the MOF particle itself is hepatoprotective, i.e., it is capable of releasing components with hepatoprotective activity. For example, fumaric acid is a bioactive molecule that can block pro-inflammatory actions and exhibit immunomodulatory, anti-inflammatory, and anti-oxidative effects. It can be used as the linking ligand in forming hepatoprotective MOF particles to protect the liver from drug-induced liver injury.

The MOFs may include a counter ion, X, in Formula (I). In a variation, the counter ion is O²⁻, sulfate, nitrate, nitrite, sulfite, bisulfite, phosphate, hydrogen phosphate, dihydrogen phosphate, diphosphate, triphosphate, phosphite, chloride, chlorate, bromide, bromate, iodide, iodate, carbonate, bicarbonate, sulfide, hydrogen sulphate, selenide, selenate, hydrogen selenate, telluride, tellurate, hydrogen tellurate, nitride, phosphide, arsenide, arsenate, hydrogen arsenate, dihydrogen arsenate, antimonide, antimonate, hydrogen antimonate, dihydrogen antimonate, fluoride, boride, borate, hydrogen borate, perchlorate, chlorite, hypochlorite, perbromate, bromite, hypobromite, periodate, iodite, or hypoiodite; or a combination thereof.

The MOFs can have pores of predefined shapes, sizes, and functionalities by choosing appropriate building blocks during synthesis of MOFs. In some forms, with a selected metal ion and a certain ligand, MOFs of the same general Formula (I) but of different structures are obtained. For example, iron-based MIL-88B and MIL-101 share the same formula but are different in their modes of ligand connection to the octahedral trimers of iron: in MIL-101, the ligands assemble in the form of rigid tetrahedra, whereas in MIL-88B, the ligands form trigonal bipyramids, enabling spacing between the trimers.

In some cases, a capping molecule is added during synthesis of MOFs to control the size of MOFs. Such a capping molecule can only coordinate to a single metal ion, which may stop the growth of MOFs.

c. Covalent Organic Frameworks

The particles may also be or contain another type of crystalline porous materials, covalent organic frameworks (COFs). COFs have repeating units resulting in a network as in the case of MOFs. The network of COFs is polymeric and contains only organic moieties (instead of organic-inorganic hybrid moieties as in the case of MOFs). Both COFs and MOFs have similar advantages including tunable features in terms of internal surface area and pore volume as well as versatile structural properties and chemical functionality. The particles based on COFs are also suitable for encapsulating hepatoprotective agents or other protective agents for preferential uptake by the liver.

3. Polymers

The short-circulating particles may be formed from or contain one or more polymers. Suitable polymers for forming the short-circulating particles include, but are not limited to, polyhydroxyesters such as polylactic acid, polyglycolic acid, and copolymers of lactic acid and glycolic acid, polycaprolactone, polyanhydrides such as polysebacic anhydride and polydioxidone, and blends or copolymers thereof. Preferably, the short-circulating particles do not contain a polymeric surface coating composed of natural polysaccharides such as dextran (Dex), polysialic acid (PSA), hyaluronic acid (HA), chitosan (CH), and heparin, or synthetic polymers such as polyvinyl pyrrolidone (PVP), polyvinyl alcohol (PVA), polyacrylamide (PAM), poly(N-isopropylacrylamide) (PNIPAM), poly(2-oxazoline), polyethylenimine (PEI), poly(acrylic acid), polymethacrylate, polyelectrolytes, polyhydroxyethylmethacrylate (poly-HEMA), polyalginic acid (alginate), poly(ethylene glycol) (PEG), poly(ethylene glycol) (PEG), polyethylene oxide (PEO), PEG-based copolymers including poloxamers, poloxamines, and polysorbates, and copolymers or blends thereof.

Exemplary polymers for forming the short-circulating particles are copolymers of lactic acid and glycolic acid (denoted as PLGA). PLGA degradation can be controlled by selecting the monomer ratio of LA and GA. PLGA with different ratios of lactic acid (LA) (which has a longer degradation time, up to one or two years) to glycolic acid (GA) (which has a short degradation time, as short as a few days to a week), can be used to form particles that provide release over a broad time frame. The preferred PLGA polymeric particles have a molecular weight from 5 to 50 kDa, from 5 to 40 kDa, from 5 to 30 kDa, from 10 to 50 kDa, from 10 to 40 kDa, or from 10 to 30 kDa. The preferred PLGA polymeric particles have a LA-to-GA copolymer ratio from 40:60 to 80:20, from 40:60 to 70:30, from 40:60 to 60:40, from 40:60 to 50:50, from 50:50 to 80:20, from 60:40 to 80:20, or from 70:30 to 80:20. The preferred PLGA polymeric particles have a size range from 200 to 1,000 nm, from 300 to 1,000 nm, from 400 to 1,000 nm, from 500 to 1,000 nm, from 600 to 1,000 nm, from 200 to 900 nm, from 200 to 800 nm, from 200 to 700 nm, from 200 to 600 nm, or from 200 to 500 nm.

4. Alternative Particles

Optionally, the short-circulating particles may be or contain lipid-based particles or silica-based mesoporous particles.

Lipid-based particles, such as liposomes, multilamellar liposomes, and solid lipid nanoparticles may contain lipid molecules with variations in polar head groups and functional chemistries, including 1,2-dilauroyl-sn-glycero-3-phosophocholine (DLPC), 1,2-dimyristol-sn-glycero-3-phosophocholine (DMPC), 1,2-dipalmitoyl-sn-glycero-3-phosophocholine (DPPC), 1,2-distearoyl-sn-glycero-3-phosophocholine (DSPC), 1,2-dioleoyl-sn-glycero-3-phosophocholine (DOPC), 1,2-dimyristol-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE), 1,2-dimyristol-sn-glycero-3-phosphate (DMPA), 1,2-dipalmitoyl-sn-glycero-3-phosphate (DPPA), 1,2-dioleoyl-sn-glycero-3-phosphate (DOPA), 1,2-dimyristol-sn-glycero-3-phosophoroglycerol (DMPG), 1,2-dipalmitoyl-sn-glycero-3-phosophoroglycerol (DPPG), 1,2-dioleoyl-sn-glycero-3-phosophoroglycerol (DOPG), 1,2-dimyristol-sn-glycero-3-phospho-L-serine (DMPS), 1,2-dipalmitoyl-sn-glycero-3-phospho-L-serine (DPPS), 1,2-dioleoyl-sn-glycero-3-phospho-L-serine (DOPS), or a combination thereof. The lipid particles may also contain various percentages of cholesterol.

Exemplary lipid-based particles and methods for making these particles are described in U.S. Pat. No. 4,588,578 to Fountain and U.S. Pat. No. 5,567,434 to Szoka.

Silica-based mesoporous nanoparticles and microparticles are readily cleared from the blood through the complement pathway and, therefore, can aid in liver accumulation/targeting. These particles may be formed from or contain glycerol-derived polyoyl-based silanes, orthosilicic acid, sodium metasilicate, tetraethyl orthosilicate, tetramethoxysilane, tetrakis (2-hydroxyethyl) orthosilicate, or a combination thereof. Exemplary suitable mesoporous silica nanoparticles (MSNs) are mobile crystalline material class materials (e.g., MCM-41 and MCM-48) and Santa Barbara amorphous-type mesoporous class materials (e.g., SBA-15), such as disclosed in Chem. Mater., 1997, 9 (10), pp. 2123-2126; J. Phys. Chem. C, 2008, 112 (46), pp 17809-17813; and J. Pharm. Sci., 2009, 98 (8), pp. 2648-2658. Suitable size ranges for the MSNs include 60 to 1,000 nm, 60 to 900 nm, 60 to 800 nm, 60 to 700 nm, 60 to 600 nm, 60 to 500 nm, 60 to 400 nm, 200 to 900 nm, 200 to 800 nm, 200 to 700 nm, 200 to 600 nm, 200 to 500 nm, or 200 to 400 nm. Pore sizes of MSNs can range from 1 to 10 nm, 1 to 9 nm, 1 to 8 nm, 1 to 7 nm, 1 to 6 nm, 1 to 5 nm, 1 to 4 nm, 2 to 10 nm, 2 to 9 nm, 2 to 8 nm, 2 to 7 nm, 2 to 6 nm, 2 to 5 nm, or 2 to 4 nm. The structures of MSNs exist in various crystalline shapes including hexagonal, cubic, and rod shapes. Both hexagonal and cubic shapes within the listed size ranges are applicable. Rod shaped MSNs within the above-listed size ranges are applicable, particularly when the aspect ratio is at or below 4.

5. Optional Particles for Differential Uptake by Liver

Optionally, long-circulating or stealth particles are administered with the short circulating particles. The long-circulating particles may also contain one or more hepatoprotective agents. The use of the long-circulating particles may delay the uptake of the hepatoprotective agent(s) in the liver to allow for a prolonged liver exposure and a long-term protection against drug-induced liver damages.

In some embodiments, the compositions may contain an additional population of long-circulating or stealth particles, which contain polymers with stealth properties or contain a polymeric surface coating with stealth properties. The long-circulating particles can evade immune recognition to have longer circulation times. The long-circulating particles may also contain one or more hepatoprotective agents. Such a composition allows for rapid uptake by the liver of the short-circulating particles followed by delayed uptake by the liver of the long-circulating particles.

The long-circulating particles may be or contain one or more polymers with stealth properties, e.g., are sufficiently flexible and hydrophilic such that they provide a hydrophilic surface. These polymers include natural polysaccharides such as dextran (Dex), polysialic acid (PSA), hyaluronic acid (HA), chitosan (CH), and heparin, as well as synthetic polymers such as polyvinyl pyrrolidone (PVP), polyvinyl alcohol (PVA), polyacrylamide (PAM), poly(N-isopropylacrylamide) (PNIPAM), poly(2-oxazoline), polyethylenimine (PEI), poly(acrylic acid), polymethacrylate, polyelectrolytes, polyhydroxyethylmethacrylate (poly-HEMA), polyalginic acid (alginate), poly(ethylene glycol) (PEG), poly(ethylene glycol) (PEG), polyethylene oxide (PEO), PEG-based copolymers including poloxamers, poloxamines, and polysorbates, and copolymers or blends thereof. In particular forms, the polymers are linear or branched PEG polymers.

Alternatively, the long-circulating particles may contain a hydrophobic or amphiphilic particle at the core, which is further coated with one or more of the aforementioned polymers with stealth properties. For example, the hydrophobic or amphiphilic particle at the core may be a PLGA-based particle. The coating may cover at least 30%, 40%, 50%, or 60% of the surface of the particle at the core on average. Up to 80%, 85%, 90%, 95%, or even 100% of the surface of the particle at the core may be covered with the coating.

6. Liver-Specific Targeting Moiety

Generally, the particles are not coated with a liver-specific targeting moiety. However, in some embodiments, the particles may be coated with a liver-specific targeting moiety. Exemplary liver-specific target moieties include asialoorasomucoid (ASOR) polypeptides, N-acetyl-galactosamine (NAG) sugars, asialotrianntenary (A3) polypeptides, arabinogalactan, mannan, and hyaluronan (HA) polypeptides. ASOR, NAG, A3, arabinogalactan or other synthetic or naturally occurring galactose-presenting molecules specifically target hepatocytes via asialoglycoprotein receptors (ASGPr), while HA, NAG or mannan specifically target liver sinusoidal endothelial cells (LSECs) via the HA, NAG or mannose receptors, respectively. Other targeting molecules include an organic surface agent such as biotin, avidin, folic acid, lipoic acid, ascorbic acid, an antibody or antibody fragment, a peptide, or a protein.

7. Size

The particles can have any suitable size. Typically the particles are nanoparticles, microparticles, or a combination thereof. The composition may contain particles that are substantially homogenous in size. In some embodiments, the composition contains particles having different sizes. Suitable particles are generally in the range of about 50 nm to about 10 μm in at least one dimension. The particle size may be the mean particle size as measured by techniques such as microscopy or light scattering.

Mean particle size generally refers to the statistical mean particle size (diameter) of the particles in the composition. Two populations can be said to have a “substantially equivalent mean particle size” when the statistical mean particle size of the first population of particles is within 20% of the statistical mean particle size of the second population of particles; more preferably within 15%, most preferably within 10%.

Microparticles generally have diameters from about 1 micron to about 100 microns, preferably from about 1 to about 50 microns, more preferably from about 1 to about 30 microns, most preferably from about 1 micron to about 10 microns. The microparticles can have any shape. Microparticles having a spherical shape are commonly referred to as microspheres.

Nanoparticles generally have a diameter, from about 1 nanometer to 1,000 nanometers, preferably from about 10 nanometers to 1,000 nanometers, more preferably from about 100 nanometers to 1000 nanometers, most preferably from about 250 nanometers to 1,000 nanometers. The nanoparticles can have any shape. Nanoparticles having a spherical shape may be referred to as nanospheres.

The particles may be MOF nanoparticles with an average diameter or at least one dimension smaller than 10,000 nm. Despite the nature of MOF to aggregate and the tendency to organize in crystal lattices of large size (e.g., microns), emulsion techniques, such as microemulsion, can be used to form MOF nanoparticles. The MOF nanoparticles may have a diameter of less than 500 nm, less than 300 nm, or less than 250 nm. However, generally the nanoparticles have diameters of at least 50 nm and up to 2,000 nm. Suitable size ranges include 250 nm to 2,000 nm, 250 nm to 1,000 nm, 250 nm to 750 nm, 200 nm to 500 nm, 200 nm to 300 nm, 250 nm to 500 nm, 250 nm to 400 nm, or 250 nm to 300 nm.

B. Active Agents

The particles contain one or more active agents to be delivered to the liver. Generally, at least one active agent is an agent that protects the liver from toxic substances (such as chemotherapeutics, alcohol, or other liver damaging entities) and their induced liver damages. One or more active agents can be encapsulated in the pores or voids of the particles, and/or can be incorporated in the framework of the particles. Alternatively, the active agents can be encapsulated or incorporated into the backbone of the particles.

In some embodiments, the materials that form the particles serve as the active agent. For example, the linking ligand in MOFs may have hepatoprotective activity.

1. Hepatoprotective Agents

The particles can contain an active agent that is a hepatoprotectant, which protects the liver from drug-induced liver damage caused by chemotherapeutic agents. The hepatoprotectant may also protect the liver from alcohol or other liver-damaging agents. The in vivo protective effect of the hepatoprotective agents can be assessed by measuring the levels of alanine aminotransferase and/or aspartate aminotransferase in the blood serum. Higher than normal levels of alanine aminotransferase and/or aspartate aminotransferase is typically correlated with liver damage. A downregulation of the levels of alanine aminotransferase and/or aspartate aminotransferase represents the protective effect of the hepatoprotective agents. For in vitro tests, the protective effect of the hepatoprotective agents can be assessed by comparing the survival rates of cultured hepatic cells with and without pretreatment or simultaneous treatment with the hepatoprotective agents, after exposure to toxic substances. The cellular levels of the biomarkers for apoptosis, including caspases (e.g., caspase 3, caspase 7, and caspase 9), can also be measured to quantify the protective effect of the hepatoprotective agents.

Suitable hepatoprotective agents include coumarin, sulforaphane, erucin, berberine, dimethyl fumarate, silibinin, and their derivatives. Additional suitable hepatoprotective agents include exemestane, amifostine, mesna, and dexrazoxane, and derivatives thereof.

“Derivative” as relates to a given compound, refers to another compound that is structurally similar, functionally similar, or both, to the specified compound. Structural similarity can be determined using any criterion known in the art, such as the Tanimoto coefficient that provides a quantitative measure of similarity between two compounds based on their molecular descriptors. Preferably, the molecular descriptors are 2D properties such as fingerprints, topological indices, and maximum common substructures, or 3D properties such as overall shape, and molecular fields. Tanimoto coefficients range between zero and one, inclusive, for dissimilar and identical pairs of molecules, respectively. A compound can be considered a derivative of a specified compound, if it has a Tanimoto coefficient with the specified compound between 0.5 and 1.0, inclusive, preferably between 0.7 and 1.0, inclusive, most preferably between 0.85 and 1.0, inclusive. A compound is functionally similar to a specified, if it induces the same pharmacological effect, physiological effect, or both, as the specified compound. “Derivative” can also refer to a modification including, but not limited to, hydrolysis, reduction, or oxidation products, of the disclosed compounds. Hydrolysis, reduction, and oxidation reactions are known in the art.

Suitable hepatoprotective agents also include chemoprotectants, such as chemoprotectants that upregulate chemoresistance of cells, particularly hepatocytes. For example, the hepatoprotective agents may be an agent that activates the nuclear receptor FXR (farnesoid X receptor) of hepatocytes, which upregulates chemoresistance genes in the cells. Activation of FXR with an agonist GW4064 increases the protection of human hepatocytes against chemotherapeutics-induced toxicity.

Suitable hepatoprotective agents also include anti-apoptotic agents. The anti-apoptotic agent may be or contain a full or partial sequence of a pro-survival Bcl-2 family protein, such as Bcl-2, Bcl-XL, Bcl-w, and MCL1, which can protect the cells against apoptosis. Optionally, the anti-apoptotic agent may be or contain an anti-apoptotic peptide. In some cases, the anti-apoptotic peptide may comprise the amino acid sequence of one or more domains of a pro-survival protein. For example, the apoptotic peptide may comprise the BH4 domain of Bcl-2 or Bcl-XL. The anti-apoptotic agent may be a caspase inhibitor, such as wedelolactone, geranylgeraniol, gambogic acid, emricasan, Gly-Phe beta-naphthylamide, tasisulam, belnacasan (VX-765), PAC-1, NSCI, NS3694, apoptosis activator 2, Z-AEVD-FMK, Z-ATAD-FMK, Z-DEVD-FMK, Z-FA-FMK, Z-IETD-FMK, Z-LEED-FMK, Z-LEHD-FMK, Z-VAD-FMK, Z-VAD(OH)-FMK, Z-VAD(OMe)-FMK, Z-VDVAD-FMK, Z-VEID-FMK, Z-VKD-FMK, Z-WEHD-FMK, Z-YVAD-FMK, Q-VD-OPH, Ac-DEVD-CHO, AZ 10417808, INF-4E, and INF 39. The anti-apoptotic agent may be a calpain inhibitor, such as calpeptin, MDL-28170 and PD150606.

Suitable hepatoprotective agents also include oligonucleotides that can inhibit apoptosis. Exemplary oligonucleotides are small interfering RNAs (siRNAs) that can target and inhibit the expression of pro-apoptotic proteins, such as BAX, BID, BAK, and BAD.

Suitable hepatoprotective agents also include anti-inflammation compounds and antioxidants commonly used as dietary supplements, nutraceuticals, or pharmaceuticals. Exemplary compounds include resveratrol, melatonin, α-tocopherol (vitamin E), ubiquinol (coenzyme Q), carnosine, ascorbic acid (vitamin C), carotenoids, glutathione, lipoic acid, uric acid, curcumin, colchicine, capsaicin, epigallocatechin-3-gallate (EGCG), and quercetin, and derivatives thereof.

The composition contains an effective amount of the hepatoprotective agent to reduce or prevent apoptosis of hepatocytes that would otherwise be induced by administering the chemotherapeutic agent alone. The terms “inhibit” and “reduce” refer to a decrease in activity or expression. This can be a complete inhibition or reduction of activity or expression, or a partial inhibition or reduction. Inhibition or reduction can be compared to a control or to a standard level. Inhibition can be a 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% reduction in activity or expression compared to a control.

An exemplary effective amount of the hepatoprotective agent in the composition for controlling, limiting, or reducing toxicity to the liver induced by chemotherapeutic agents is between about 1 and about 200 mg, between about 1 and about 100 mg, between about 1 and about 50 mg, between about 1 and about 20 mg, between about 5 mg to about 200 mg, between about 5 mg to about 100 mg, between about 5 mg to about 50 mg, between about 5 mg to about 20 mg, between about 10 mg to about 200 mg, between about 10 mg to about 100 mg, between about 10 mg to about 50 mg, between about 10 mg to about 20 mg, between about 20 mg to about 200 mg, between about 20 mg to about 100 mg, or between about 20 mg to about 50 mg, per kilogram of a patient or subject per week. When particles containing an effective amount of the hepatoprotective agent are administered with, prior to, or following the administration of a chemotherapeutic agent, the dosage of the chemotherapeutic agent for administration may increase by at least 5%, 10%, 20%, 30%, 40%, 50%, 60%, 70%, 80%, 90%, 100%, 200%, or 300% without causing increased toxicity to the liver, as measured by the level of alanine aminotransferase or/and aspartate aminotransferase in blood serum, compared to administering the chemotherapeutic agent alone. In some embodiments, the dosage of the chemotherapeutic agent, when administered in combination with a composition delivering hepatoprotective agents with preferential accumulation in the liver, is at least 5%, 10%, 20%, 30%, 40%, 50%, 60%, 70%, 80%, 90%, 100%, 200%, or 300% higher than the maximum recommended starting dose of the chemotherapeutic for a first-in-human clinical trial accepted by the U.S. Food and Drug Administration.

In some embodiments, the particles are formed from MOFs, and the linking ligand in the MOFs may be or contain a hepatoprotective agent. For example, the MOF may contain fumaric acid, and fumaric acid may be the hepatoprotective agent.

Particles containing a hepatoprotective agent for the liver may be administered to a subject along with or in advance of a chemotherapeutic agent for treatment of symptoms of tumor or cancer in a tissue, particularly tissues other than liver. The particles containing a hepatoprotective agent generally reduce apoptosis of hepatocytes that are otherwise induced when administering the chemotherapeutic agent alone. Further, the particles containing the hepatoprotective agent do not interfere with the efficacy of the chemotherapeutic agent against the tumor.

2. Additional Active Agents

In some embodiments, in addition to hepatoprotective agents, the particles or the composition also contain a compound or molecule that can be used to treat a disease or complication of the liver. A compound or molecule that can be used to treat a disease or complication of the liver include, for example, a polypeptide, a nucleic acid molecule (e.g., a construct encoding a polypeptide, or an antisense RNA, RNAi, or siRNA nucleic acid molecule), an antiviral agent, a drug or small molecule (e.g., ursodeoxycholic acid and its amino acid conjugate, tauroursodeoxycholic acid and glycourodeoxycholic acid; s-adennosyl-L-methionine; 1-(isopropylamino) 3-(naphthalen-1-yloxy)propan-2-ol; hydroxyurea; or cortocosteriods), or an anti-apoptotic agent.

Exemplary antiviral drugs suitable for inclusion in the particles or compositions include Acyclovir, Adefovir, Amantadine, Amprenavir, Ampligen, Arbidol, Atazanavir, Atripla, Balavir, Boceprevirertet, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Stavudine, Tea tree oil, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine.

Exemplary antibiotics suitable for inclusion in the particles or compositions include members of the groups of Tetracyclines, Sulfonamides, Quinolones, Penicillin combinations, Penicillins, Oxazolidonones, Nitrofurans, Monobactams, Macrolides, Lincosamides, Cephalosporins, Carbapenems, Ansamycins, and Aminoglycosides.

Exemplary antifungal drugs suitable for inclusion in the particles or compositions include Clotrimazole, Posaconazole, Ravuconazole, Econazole, Ketoconazole, Voriconazole, Fluconazole, Itraconazole, Tebuconazole and Propiconazole. In another embodiment, the additional therapeutic agent is an echinocandin. Representative echinocandins include, but are not limited to pneumocandins, Echinocandin B, Cilofungin, Caspofungin, Micafungin (FK463), and Anidulafungin (VER-002, V-echinocandin, LY303366).

3. Loading of Active Agents in Particles

The particles can contain at least 2%, 5%, 10%, 11%, 12%, 13%, 14%, 15% (w/w) or even greater amounts of a hepatoprotective agent. For example, the particles can encapsulate a hepatoprotective agent in a range of 5-15%, 10-15%, 10-20%, 10-30%, 10-40%, 5 to 40%, or 10-50% (w/w). In some embodiments, a preferred loading range of a hepatoprotective agent in the particles is between 10% and 30% (w/w).

In some forms, a hepatoprotective agent is encapsulated or incorporated in the particles at a weight percentage of at least 2%, 5%, 6%, 7%, 8%, 9%, 10%, 11%, 12%, 13%, 14%, or 15%. Optionally the particles are formed from or contain an MOF. Optionally, the particles are MOF particles having an average diameter or at least one dimension that is between about 50 nm and 10 μm and contain a hepatoprotective agent encapsulated or incorporated in the particles at a weight percentage of at least 5%, 6%, 7%, 8%, 9%, 10%, 11%, 12%, 13%, 14%, or 15%.

The particles may be polymeric particles, such as prepared with PLGA, and have an average diameter or at least one dimension between about 200 nm and 10 μm and contain a hepatoprotective agent encapsulated or incorporated therein at a weight percentage of at least 5%, 6%, 7%, 8%, 9%, 10%, 11%, 12%, 13%, 14%, or 15%.

C. Pharmaceutical Excipients

The composition may also contain one or more pharmaceutically acceptable excipients. Representative excipients include solvents, diluents, carriers, pH modifying agents, preservatives, antioxidants, suspending agents, wetting agents, viscosity modifiers, tonicity agents, stabilizing agents, and combinations thereof. Suitable pharmaceutically acceptable excipients are for example, selected from materials which are generally recognized as safe (GRAS), and may be administered to an individual without causing undesirable biological side effects or unwanted interactions.

Excipients can be added to the composition to assist in sterility, preservations, and to adjust and/or maintain pH or isotonicity. Particles for delivering hepatoprotective agents or other active agents for protection of the liver can be suspended in sterile saline, phosphate buffered saline (PBS), balanced salt solution (BSS), viscous gel or other pharmaceutically acceptable carriers for administration. The composition may also be a sterile solution, suspension, or emulsion in a nontoxic, parenterally acceptable diluent or solvent.

1. Excipients for Liquid Formulations

Generally, the composition is in a liquid form, as a liquid formulation suitable for administration to a patient.

The liquid pharmaceutical carrier can include one or more physiologically compatible buffers, such as a phosphate buffers. One skilled in the art can readily determine a suitable saline content and pH for an aqueous carrier for administration.

Liquid formulations may include one or more suspending agents, such as cellulose derivatives, sodium alginate, polyvinylpyrrolidone, gum tragacanth, or lecithin. Liquid formulations may also include one or more preservatives, such as ethyl or n-propyl p-hydroxybenzoate.

In some embodiments the liquid formulation may contain one or more solvents that are low toxicity organic (i.e., nonaqueous) class 3 residual solvents, such as ethanol, acetone, ethyl acetate, tetrahydofuran, ethyl ether, and propanol. Any such solvent included in the liquid formulation should not detrimentally react with the one or more active agents present in the liquid formulation. Solvents such as freon, alcohol, glycol, polyglycol, or fatty acid, can also be included in the liquid formulation as desired to increase the volatility of the solution or suspension.

Liquid formulations may also contain minor amounts of polymers, surfactants, or other excipients well known to those of the art. In this context, “minor amounts” means an amount that is sufficiently small to avoid adversely affecting uptake of the particles by the liver, in the case of compositions and formulations containing a hepatoprotective agent, or to avoid adversely affecting uptake of a chemotherapeutic agent by the tumor tissue, in the case of formulations containing a chemotherapeutic agent.

D. Forms of the Composition

Generally, the composition is in a liquid form, as a liquid formulation suitable for administration to a patient. The liquid formulation is typically isotonic relative to physiological fluids and of approximately the same pH, ranging e.g., from about pH 4.0 to about pH 7.4, more preferably from about pH 6.0 to pH 7.0.

The liquid formulation may be an emulsion or a suspension.

Alternatively, the composition may be in the form of a gel, a paste, or a capsule which contains the particles encapsulated therein (in solid or liquid form).

Alternatively, the composition can be a solid, for example a powder obtained by lyophilization of a liquid composition. The solid can be reconstituted with an appropriate carrier or diluent prior to administration. When in the solid form, the composition may be in the form of a pill, a capsule, a powder, a cake, or a pressed shape.

The powder and liquid compositions can be used to form aerosol formulations, e.g., a fine mist of particles, which can be in a suspension, whether or not it is produced using a propellant.

E. Combination Formulation

In some cases, the composition is a combined formulation which contains both a hepatoprotective agent and another active agent. In some embodiments, the active agent is a chemotherapeutic agent for treatment of tumor or cancer, particularly in tissues other than liver. Preferably, the composition contains an effective amount of the hepatoprotective agent to reduce or prevent apoptosis of hepatocytes that would otherwise be induced by administering the chemotherapeutic agent alone. When particles containing an effective amount of the hepatoprotective agent are administered with, prior to, or following the administration of a chemotherapeutic agent, the dosage of the chemotherapeutic agent for administration may increase by at least 5%, 10%, 20%, 30%, 40%, 50%, 60%, 70%, 80%, 90%, 100%, 200%, or 300% without causing any increased toxicity to the liver, as measured by the levels of alanine aminotransferase or/and aspartate aminotransferase in blood serum, compared to administering the chemotherapeutic agent alone. In some embodiments, the dosage of the chemotherapeutic agent, when administered in combination with a composition delivering a hepatoprotective agent with preferential accumulation in the liver, is at least 5%, 10%, 20%, 30%, 40%, 50%, 60%, 70%, 80%, 90%, 100%, 200%, or 300% higher than the maximum recommended starting dose of the chemotherapeutic agent for a first-in-human clinical trial accepted by the U.S. Food and Drug Administration.

The combined formulation may contain two or more populations of particles, where the populations of particles are different from each other with respect to one or more properties, such as surface properties, chemical compositions, average particle sizes or greatest dimensions, and/or particle size distributions. The first population of particles may contain a hepatoprotective agent. The second population of particles may contain a chemotherapeutic agent, and the second population of particles may have a more hydrophilic surface compared to the surface of the first population of particles.

The combined formulation may contain at least one type of particles for delivery into the liver and at least a second, different type of particles for delivery of a chemotherapeutic agent to a tissue other than the liver.

### II. System for Chemotherapy

The particles containing a hepatoprotective agent may be administered to a patient prior to or along with, or following a chemotherapeutic agent. For example, the particles containing a hepatoprotective agent may be included in a system for chemotherapy. The system can contain two or more formulations. Typically a first formulation contains the hepatoprotective agent, and a second formulation contains the chemotherapeutic agent. Preferably, the system includes particles containing an effective amount of the hepatoprotective agent to reduce or prevent apoptosis of hepatocytes that would otherwise be induced by administering the chemotherapeutic agent at a given dosage alone, as exemplarily measured by the levels of alanine aminotransferase or/and aspartate aminotransferase in blood serum; or particles containing an effective amount of the hepatoprotective agent to limit, maintain, or have a detectably indistinguishable toxicity to the liver when the chemotherapeutic agent is administered at an increased dosage compared to that induced by administering the chemotherapeutic agent alone at the original dosage. When particles containing an effective amount of the hepatoprotective agent are administered with, prior to, or following the administration of a chemotherapeutic agent, the dosage of the chemotherapeutic agent for administration may increase by at least 5%, 10%, 20%, 30%, 40%, 50%, 60%, 70%, 80%, 90%, 100%, 200%, or 300% without causing any increased toxicity to the liver compared to administering the chemotherapeutic agent alone. In some embodiments, the dosage of the chemotherapeutic agent, when administered in combination with a composition delivering one or more hepatoprotective agents with preferential accumulation in the liver, is at least 5%, 10%, 20%, 30%, 40%, 50%, 60%, 70%, 80%, 90%, 100%, 200%, or 300% higher than the maximum recommended starting dose of the chemotherapeutic for a first-in-human clinical trial accepted by the U.S. Food and Drug Administration.

A. First Formulation Containing Hepatoprotective Particle

The first formulation in the system may contain particles for preferential delivery and quick accumulation in liver to deliver or release one or more hepatoprotective agents. The first formulation may include short-circulating particles that contain a hepatoprotectant. For example, the first formulation may include MOF particles having an average diameter or at least in one dimension that is between about 50 nm and 10 μm with a hepatoprotective agent, such as sulforaphane, encapsulated or incorporated in the MOF at a weight percentage of at least 5%, 6%, 7%, 8%, 9%, 10%, 11%, 12%, 13%, 14%, or 15%.

The first formulation may contain particles for preferential accumulation in liver to deliver other active agents for protecting the liver against or for treatment of one or more diseases or disorders of the liver or one or more symptoms associated with these diseases or disorders. Optionally, these particles are included in the same formulation as the hepatoprotective agent.

The first formulation may contain two or more populations of particles for differential uptake by the liver, where the different populations of particles have different solubility, different surface hydrophobicity/hydrophilicity, different dimensions/sizes, different structures, and/or different particle size distributions. Optionally the first formulation includes a first population of particles having a hydrophobic surface and a second population of particles having a more hydrophilic surface.

B. Second Formulation Containing Chemotherapeutic Agent

The second formulation in the system may contain a chemotherapeutic agent. The chemotherapeutic agent may be encapsulated, or it may be unencapsulated. The second formulation may contain an effective amount of the chemotherapeutic agent to kill cancer cells and/or inhibit the growth of tumors.

Particles in the second formulation that encapsulate the chemotherapeutic agent may be taken up more slowly by the liver than particles in the first formulation, which encapsulate the hepatoprotective agent. The second formulation may contain a population of particles that has a slower liver uptake rate compared to the particles in the first formulation.

1. Chemotherapeutic Agents

Exemplary chemotherapeutic agents include sorafenib, dacarbazine, erlotinib hydrochloride, cisplatin, cetuximab, sunitinib, doxorubicin, daunorubicin, bevacizumab, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab, rituximab, and combinations thereof. The majority of chemotherapeutic agents are divided into: alkylating agents, antimetabolites, anthracyclines, plant alkaloids, topoisomerase inhibitors, and other antitumor agents.

2. Excipients

The second formulation may also contain one or more pharmaceutically acceptable excipients. Representative excipients are the same as the excipients listed above with respect to the compositions containing the hepatoprotective agent. For example, chemotherapeutic agents can be suspended in sterile saline, water, phosphate buffered saline (PBS), balanced salt solution (BSS), Ringer's solution, a viscous gel, or other pharmaceutically acceptable carriers for administration.

C. Administration of the Formulations

The first and second formulations may be administered simultaneously or sequentially.

The first formulation containing particles containing hepatoprotective agent and the second formulation containing a chemotherapeutic agent may be combined prior to administration for administration concomitantly, e.g., as an admixture. Alternatively, the first formulation containing particles containing a hepatoprotective agent and the second formulation containing a chemotherapeutic agent are administered separately and simultaneously, e.g., via separate intravenous lines into the same subject. In some embodiments, the first formulation containing a hepatoprotective agent is administered at a same frequency and/or on the same schedule as the second formulation containing a chemotherapeutic agent. In other embodiments, the first formulation containing a hepatoprotective agent is administered less frequently than the second formulation. Optionally, the first formulation is administered prior to (e.g., within the same day as, or minutes, hours, or days in advance) the second formulation. However, it is generally unnecessary to administer the first formulation containing a hepatoprotective agent for weeks, such as three weeks, prior to administering the second formulation containing a chemotherapeutic agent.

Alternatively, the first and second formulations are administered sequentially. For example, the first formulation containing particles containing a hepatoprotective agent is administered first followed by the second formulation containing a chemotherapeutic agent. Optionally the first formulation containing a hepatoprotective agent may be administered again following the administration of the chemotherapeutic agent. The time between administering the first formulation and administering the second formulation may be minutes, such as at least 10 minutes, at least 30 minutes, or up to 1 hour; hours, such as 1 hour, 2 hours, 2 to 10 hours, or up to 24 hours; days, such as 1 day, 2 days, or up to two weeks.

In one embodiment, the system contains a first formulation containing MOF particles encapsulating a hepatoprotective agent, such as sulforaphane, and a second formulation containing a chemotherapeutic agent, such as doxorubicin. The first formulation may be administered prior to administration of the second formulation. Alternatively, the first formulation may be administered concomitantly or simultaneously with the second formulation.

### III. Methods of Making Particles

A. MOF-Based Particles

The synthesis of MOF-based particles is generally performed in the presence of energy, which may be supplied, for example, by heating, for instance under hydrothermal or solvothermal conditions, but also by microwave, by ultrasound, by grinding, by a process involving a supercritical fluid, etc.

Nonlimiting examples of protocols that may be used for hydrothermal or solvothermal conditions are described, for example, in K. Byrapsa, et al. “Handbook of hydrothermal technology”, Noyes Publications, Parkridge, N.J., USA, William Andrew Publishing, LLC, Norwich, N.Y., USA, 2001. In hydrothermal or solvothermal techniques, crystals slowly grow from a hot solution of metal precursor. Hydro/solvothermal synthesis can be carried out under dynamic or static, ambient or autogenous pressure conditions.

For example, a typical solvothermal synthesis involves a solution of metal ions (e.g., FeCl₃.6H₂O) and a ligand, (e.g., fumaric acid) in a solvent (e.g., dimethylformamide, absolute ethanol, methanol, or distilled water), placed into a suitable vessel, such as a Teflon-lined steel flask. Optionally acetic acid is added to adjust the pH or to provide a modulator effect, i.e., slow down the crystal growth rate, thus avoiding fast precipitation of amorphous product (Schaate A, et al., Chem. Eur. J., 2011, 17(24):6643-6651; Hu Z, et al., Inorg. Chem., 2015, 54(10):4862-4868). The solution is autoclaved at about 100° C. for one or more times (e.g., at 2, 6, or 24 hours). Obtained precipitate is recovered by centrifugation, e.g., at 10,000 rpm (5,600×g) for 10 minutes.

Optionally microwave irradiation or ultrasonic energy assist the synthesis. For the synthesis via microwaves, non-limiting examples of protocols that may be used are described, for example, in G. Tompsett, et al. ChemPhysChem, 2006, 7, 296; in S. E. Park, et al. Catal. Survey Asia, 2004, 8, 91; in C. S. Cundy, Collect. Czech. Chem. Comm., 1998, 63, 1699; or in S. H. Jhung, et al. Bull. Kor. Chem. Soc., 2005, 26, 880. Microwave irradiation may assist hydro/solvothermal synthesis in faster crystallization times, phase selectivity, narrower particle size distributions, and often a control of the morphology.

The hydrothermal or solvothermal synthesis typically occurs at a temperature in the range of 0 to 220° C. and is generally performed in glass (or plastic) containers when the temperature is below the boiling point of the solvent. When the temperature is higher or when the reaction is performed in the presence of fluorine, Teflon bodies inserted into metal bombs are typically used.

The solvents used in the synthesis of MOFs are generally polar. Exemplary solvents include water, alcohols, dimethylformamide, dimethyl sulfoxide, acetonitrile, tetrahydrofuran, diethylformamide, chloroform, cyclohexane, acetone, cyanobenzene, dichloromethane, nitrobenzene, ethylene glycol, dimethylacetamide, or mixtures of these solvents. One or more cosolvents may also be added at any step in the synthesis for better dissolution of the compounds of the mixture. For example, the cosolvents may be monocarboxylic acids, such as acetic acid, formic acid, benzoic acid, etc.

Monocarboxylic acids can be added as cosolvents. Besides having a solubilizing effect, monocarboxylic acids also makes it possible to stop the crystal growth of the MOFs because they can only coordinate one iron ion. As a result, the growth of the crystal network is slowed down, and then stopped. Therefore, the addition of a monocarboxylic cosolvent, such as acetic acid, formic acid, benzoic acid, etc., makes it possible to reduce the size of the MOF particles obtained. A monocarboxylic cosolvent can be added to promote the production of nanoparticles (particle size <1 μm).

Another method of preparing crystalline MOFs involves the addition of a solution of a metal salt to a solution containing an appropriate blend of ligands, some of which contain multidentate functional groups, and others of which contain monodentate functional groups, in the presence of a suitable templating agent.

Alternatively, reverse-phase emulsion techniques are used to prepare MOF particles. The cationic cetyltrimethylammonium bromide (CTAB)/isooctane/1-hexanol/water system provides control over the particle size.

The solvents used during synthesis may remain in the pores of the materials and are generally be removed by one or more appropriate techniques such as heating to activate MOFs after synthesis. Alternatively to remove any free organic acid in the pores of the MOF particles, the recovered precipitate or solid mass from synthesis can be suspended in ethanol or deionized water, generally under stirring, for a period of time such as overnight or 1-10 hours. Centrifuging separates the MOF particles from the supernatant containing removed organic acid. The MOF particles can be further dried under air or vacuum.

B. Polymeric Particles

Suitable techniques for making short-circulating polymeric particles include, but are not limited to, solvent evaporation, solvent removal, spray drying, phase inversion, low temperature casting, and nanoprecipitation. The hepatoprotective agent, other active agent for the protection of the liver, and/or pharmaceutically acceptable excipients, including pH modifying agents, disintegrants, preservatives, and antioxidants, can optionally be incorporated into the particles during particle formation.

1. Emulsion or Solvent Evaporation

In this method, the polymer(s) are dissolved in a volatile organic solvent, such as methylene chloride. The organic solution containing the polymer is then suspended in an aqueous solution that contains an emulsifier, e.g., a surfactant agent such as poly(vinyl alcohol) typically under probe sonication for a period of time (e.g., 2 minutes) to form an emulsion. The hepatoprotective agent and other components of the particles may be dissolved in the organic solvent with the polymer or in the aqueous solution, depending on its hydrophilicity/hydrophobicity. The emulsion is added to another large volume of the emulsifier with magnetic stirring to evaporate the organic solvent. The resulting emulsion is stirred until most of the organic solvent evaporated, leaving solid particles. The resulting particles are washed with water and dried overnight in a lyophilizer. Particles with different sizes and morphologies can be obtained by this method.

2. Solvent Removal

In this method, the polymer, the hepatoprotective agent and other components in the particles are dispersed or dissolved in a suitable solvent. This mixture is then suspended by stirring in an organic oil (such as silicon oil) to form an emulsion. Solid particles form from the emulsion, which can subsequently be isolated from the supernatant.

3. Spray Drying

In this method, the polymer, the hepatoprotective agent and other components of the particles are dispersed or dissolved in a suitable solvent. The solution is pumped through a micronizing nozzle driven by a flow of compressed gas, and the resulting aerosol is suspended in a heated cyclone of air, allowing the solvent to evaporate from the microdroplets, forming particles.

4. Phase Inversion

In this method, the polymer, the hepatoprotective agent and other components of the particles are dispersed or dissolved in a “good” solvent, and the solution is poured into a strong non-solvent for the polymeric components to spontaneously produce, under favorable conditions, nanoparticles or microparticles.

5. Low Temperature Casting

Methods for very low temperature casting of particles are described in U.S. Pat. No. 5,019,400 to Gombotz et al. In this method, the polymer, the hepatoprotective agent and other components of the particles are dispersed or dissolved is a solvent. The mixture is then atomized into a vessel containing a liquid non-solvent at a temperature below the freezing point of the solution which freezes the polymer, the hepatoprotective agent and other components of the particles as tiny droplets. As the droplets and non-solvent for the components are warmed, the solvent in the droplets thaws and is extracted into the non-solvent, hardening the particles.

C. Forms for Administration

Optionally, after particle formation, size separation, if necessary, and/or washing and drying, of the particles occurs.

Optionally, the powder may be added to a suitable carrier at the time of administration. Optionally, one or more excipients are added to form a suspension for administration to a patient.

Alternatively, the particles may be encapsulated in a capsule or formed into a solid dosage form.

### IV. Methods of Using the Particles

Generally the system containing both formulations, i.e., a chemotherapeutic agent formulation and a particle formulation for preferential delivery to the liver of a hepatoprotective agent or another active agent for the protection of the liver, is used to treat a subject or a patient having a solid, malignant tumor. For example, the subject or the patient may have or be at risk of developing brain cancer, breast cancer, bone cancer, lungs cancer, prostate cancer, pancreatic cancer, cervical cancer, colon cancer, leukemia, or lymphoma. In a preferred embodiment the subject does not have liver cancer, liver tumor, or liver metastases, or is not being treated for liver cancer.

The system can be administered to a patient in an effective amount to prevent or diminish hepatotoxic adverse effects induced by chemotherapy, such as preventing or diminishing mononuclear infiltration of sinusoids.

A. Adjunctive for Chemotherapy

The composition containing the particles containing a hepatoprotective agent for the protection of the liver may be administered to a subject along with or prior to administering a chemotherapeutic agent. The particles containing the hepatoprotective agent are quickly taken up by the liver and accumulate in liver. The particles generally have a short circulation time. The composition contains an effective amount of the hepatoprotective agent to reduce or prevent apoptosis of hepatocytes that would otherwise be induced by administering the chemotherapeutic agent alone. The particles containing a hepatoprotective agent are taken up by the liver in a sufficient amount, i.e., at least 5%, 10%, 15%, 20%, 30%, 40%, 50%, 60%, 70%, or 80% of the particles are taken up by the liver, and do not deliver the hepatoprotective agent to other tissues in an amount that interferes with the efficacy of the chemotherapeutic agent against tumors in the other tissues.

Higher doses of chemotherapeutic agents can be administered safely and effectively to a patient using the systems described herein compared to when the same chemotherapeutic agent is administered in the absence of the hepatoprotective composition. For example, the dose may be 5%, 10%, 20%, 30%, 40%, 50%, 60%, 70%, 80%, 90%, 100%, 200%, or 300% higher, without showing detectably increased damage to the liver. In some embodiments, the dosage of the chemotherapeutic agent, when administered in combination with a composition containing a hepatoprotective agent with preferential accumulation in the liver, is at least 5%, 10%, 20%, 30%, 40%, 50%, 60%, 70%, 80%, 90%, 100%, 200%, or 300% higher than the maximum recommended starting dose of the chemotherapeutic for a first-in-human clinical trial accepted by the U.S. Food and Drug Administration.

1. Simultaneous Delivery

In some embodiments, a combined formulation containing both the hepatoprotective agent and a chemotherapeutic is administered to the patient. In other embodiments, a first formulation containing particles containing the hepatoprotective agent and a second formulation containing a chemotherapeutic agent are combined prior to administration and administered simultaneously as an admixture. In other embodiments, a first formulation containing particles containing the hepatoprotective agent and a second formulation containing a chemotherapeutic agent are administered separately, but simultaneously, e.g., via separate intravenous lines into the same subject.

2. Administration Prior to Chemotherapeutic Agent

Alternatively, a first formulation containing particles containing the hepatoprotective agent and a second formulation containing a chemotherapeutic agent are administered sequentially, e.g., the first formulation is administered first followed by the administration of the second formulation. Optionally, the first formulation is administered again following administration of the second formulation

The time period between administering the first formulation and administering the second formulation may be minutes, such as at least 10 minutes, at least 30 minutes, or up to 1 hour; hours, such as 1 hour, two hours, 2 to 10 hours, 24 hours; days, 1 day, 2 days, or up to two weeks.

Particles containing a hepatoprotective agent or another active agents for protection of the liver can be administered in a suitable composition via any suitable route of administration, e.g., locally or systemically to the subject. Optionally, the particles may be coated or incorporated onto or into a device.

3. Bolus Administration

The composition containing particles encapsulating or containing a hepatoprotective agent can be administered in bolus and provide controlled or sustained release of the hepatoprotective agent in the liver over a period of time ranging from hours to days, such as over 5 hours, 10 hours, 20 hours, 24 hours, two days, three days, four days, five days, six days, seven days, 14 days, or 21 days. This allows a single administration of the composition containing the hepatoprotective agent to be administered and deliver an effective amount of the hepatoprotective agent to the liver to protect the liver from toxicity induced by chemotherapeutics for at least one day, two days, three days, four days, five days, six days, even seven, 14, or 21 days following administration of the composition.

B. Liver Protection

The compositions containing particles containing an active agent can be administered to a patient in need of such treatment in an effective amount to protect the liver from toxic agents or effects from such agents or to repair damage to one or more types of cells in the liver, such as hepatocytes, sinusoidal endothelial cells, Kupffer cells, hepatic stellate cells, and/or intrahepatic lymphocytes. The particles may be or contain a MOF, as described above with respect to use with the administration of cytotoxic chemotherapeutic agents. For example, MOF may contain a metal ion, such as Fe³⁺, Fe²⁺, Zn²⁺, Na⁺, Al³⁺, K⁺, Mg²⁺, and Ca²⁺. Alternatively, the MOF may contain other metal ions, including Li⁺, Rb⁺, Sr²⁺, Ba²⁺, Sc³⁺, Ti⁴⁺, Zr⁴⁺, Ta³⁺, Cr³⁺, Mo³⁺, W³⁺, Mn³⁺, Ru³⁺, Ru²⁺, Os^{3+,} Os^{2+,} Co^{3+,} Co²⁺, Ni²⁺, Ni⁺, Pd²⁺, Pd⁺, Pt²⁺, Pt⁺, Cu²⁺, Cu⁺, Au⁺, Ga³⁺, In³⁺, Si⁴⁺, Si²⁺, Ge⁴⁺, Ge²⁺, Sn⁴⁺, Sn²⁺, Bi⁵⁺, Bi³⁺, Mn²⁺, and Cr³⁺. The MOF may contain a linker, such as fumaric acid, trimesic acid, terephthalic acid, citric acid, malic acid, tartaric acid, methanoic acid, ethanoic acid, propanoic acid, butanoic acid, valeric acid, caproic acid, caprylic acid, capric acid, lauric acid, mylistic acid, palmitic acid, stearic acid, oleic acid, linoleic acid, linolenic acid, cyclohexanecarboxylic acid, phenylacetic acid, benzoic acid, oxalic acid, malonic acid, succinic acid, glutaric acid, adipic acid, pimelic acid, suberic acid, maleic acid, phthalic acid, isophthalic acid, hemimellitic acid, trimellitic acid, succinic anhydride, maleic anhydride, phthalic anhydride, glycolic acid, lactic acid, hydroxybutyric acid, mandelic acid, glyceric acid, gallic acid, malic acid, 3,3′,5,5′-azobenzene tetracarboxylic acid, zoledronic acid, tartaric acid, citric acid, ascorbic acid, imidazole, 1-methylimidazole, 2-methylimidazole, 4-methylimidazole, or γ-cyclodextrin, or derivatives thereof.

The effective amount of the compositions may be effective to treat or diminish an adverse or condition in the liver, which is associated with a disease or disorder in the patient. The diseases or conditions in the liver can be liver cancer, a metastatic cancer in the liver, a pro-oncogenic liver condition, a precancerous liver condition, hepatocellular carcinoma, cirrhosis, a post-cancer liver condition, non-alcoholic fatty liver disease (NAFLD), fatty liver, liver inflammation, liver fibrosis, non-alcoholic steatohepatitis (NASH), alcoholism-induced damage, cryptogenic cirrhosis, hepatocelullar carcinoma, liver decompensation, steatohepatitis, or chemoresistance.

Examples of agents other than chemotherapeutic agents that may induce the above-mentioned adverse liver conditions include, but are not limited to, alcohol, acetaminophen, angiotensin-converting enzyme (ACE) inhibitors, or any of the prescription, over-the-counter, or herbal entities listed in the NIH Liver Tox database (livertox.nih.gov), or combinations thereof.

The active agent can be any hepatoprotective agent, as described above.

In these embodiments, the particles or compositions containing a hepatoprotective agent can be administered prior to, simultaneous with, or following the administration of a second acid agent other than a chemotherapeutic agent, which is known to cause an adverse liver condition in an effective amount to prevent or reduce the adverse liver condition.

The particles containing a hepatoprotective agent for the liver may be administered to a patient prior to, along with, or following the administration of a non-chemotherapeutic agent that may induce an adverse effect on the liver. For example, the particles containing a hepatoprotective agent may be included in a system for delivering the non-chemotherapeutic agent. The system can contain two or more formulations. Typically a first formulation contains the hepatoprotective agent, and a second formulation contains the non-chemotherapeutic agent. The first formulation can be the same as described above. However, the dosage of the hepatoprotective agent may be varied as needed, depending on the active agent in the second formulation.

Preferably, the system includes particles containing an effective amount of the hepatoprotective agent to reduce or prevent apoptosis of hepatocytes that would otherwise be induced by administering the non-chemotherapeutic active agent at a given dosage alone, as exemplarily measured by the levels of alanine aminotransferase or/and aspartate aminotransferase in blood serum. It may be possible to administer higher dosages of the non-chemotherapeutic active agent when administered in the system or in combination with the first formulation. For example, the first formulation or particles containing the hepatoprotective agent may contain an effective amount of the hepatoprotective agent to limit, maintain, or have a detectably indistinguishable toxicity to the liver when the non-chemotherapeutic agent is administered at an increased dosage compared to that induced by administering the non-chemotherapeutic agent alone at an un-increased dosage.

When particles containing an effective amount of the hepatoprotective agent are administered with, prior to, or following the administration of a non-chemotherapeutic agent that induces adverse effects on the liver, the non-chemotherapeutic agent may be administered at its standard approved dosages or even at higher dosages while decreasing toxicity to the liver compared to administering the non-chemotherapeutic agent alone.

In some embodiments, prior to or simultaneously with drinking an alcoholic beverage, a formulation containing the particles or compositions containing a hepatoprotective agent can be administered to the individual, where the hepatoprotective agent is present in an effective amount to prevent or reduce the damage to the liver that would otherwise be caused by drinking the alcoholic beverage in the absence of the hepatoprotective agent.

The present invention will be further understood by reference to the following non-limiting examples.

### EXAMPLES

### Example 1. MOF Particles Loaded with Sulforaphane Mitigate Doxorubicin-Induced Liver Damage

Materials and Methods

Preparation of Sulforaphane-Loaded MOFs

Iron carboxylate MOF MIL-100 was prepared as described in Eur. J. Inorg. Chem., 2012, 5165-5174. Briefly, iron(III) chloride hexahydrate (6 mM) and trimesic acid (4 mM) were dissolved together in water and heated to 130° C. over 30 seconds and held at this temperature for 5.5 minutes to react. The reaction was then cooled to room temperature and centrifuged at 2,500×g for 25 minutes. The resulting solid was collected.

Iron fumarate MOF MIL-88A was prepared as described in J. Mater. Chem., 2011, 21, 2220-2227. Briefly, a solution of iron(III) chloride hexahydrate (1 mmol) and fumaric acid (1 mmol) in 5 mL of distilled water was placed into a round bottom flask and kept under magnetic stirring at 65° C. for 6 hours. The resulting solid was recovered by centrifugation at 10,000 rpm (5,600×g) for 10 min. In order to remove the free organic acid from the pores, 200 mg of the as-synthesized solid was suspended overnight into 100 mL of deionized water under stirring at room temperature. Then, the solid was recovered by centrifugation at 10,000 rpm for 10 min and dried under air.

Another iron carboxylate MOF MIL-88B was prepared as described in J. Am. Chem. Soc. 2011, 133, 17839-17847. Briefly, MIL-88B particles with modified terephthalic acids of the formula C₆H₄₋ₓRₓ (CO₂H)₂, where R═NH₂, were synthesized under either hydro or solvothermal conditions.

MOFs were lyophilized for 24 hours before use. MOFs were dissolved in a sulforaphane (SFN) solution in ethanol, where the SFN solution had a concentration of 17 mg SFN per mL of ethanol, and stirred for 16 hours at room temperature. The powder was collected by centrifugation (15 min, 20,000×g, room temperature), rapidly washed with distilled water and then collected again by centrifugation (10 min, 20,000×g, room temperature) followed by lyophilization for 24 hours.

Measurement of SFN Loading Efficacy

Loading efficacy of the hepatoprotectant SFN was determined using HPLC equipped with a UV-detector (Waters 1525). A mixture of water-acetonitrile at a 60:40 v/v ratio was used as a mobile phase. Chromatography was performed using reversed-phase Luna C18 column (Waters) at a flow rate of 1.0 mL/min and detection wavelength of 238 nm. To obtain calibration curve, SFN standard solutions in ethanol were prepared with concentrations of 500, 250, 125, 62 and 31 μg/mL. Samples for determination of the loading efficiency were prepared by suspending lyophilized SFN-loaded MOF particles in ethanol, and stirring for 1 hour in order to solubilize and wash out encapsulated SFN. After extraction of SFN from the MOF particles, the ethanol phase was collected by centrifugation (15 min, 20,000×g, room temperature). The supernatant was filtered through 0.22 am filters and the supernatant was assayed in HPLC measurements.

Measurement of SFN Release Profile

Lyophilized SFN-loaded MOF particles were dissolved in distilled water or 5 mM HEPES buffer (pH 7.4). The liquid phase was collected at 15 min, 30 min, 1 hour, and 3 hours and centrifuged (15 min, 20,000×g, room temperature). The supernatant after the centrifugation was assayed under HPLC to determine the release amount at each collection time point.

Effect of SFN on Phase 2 Enzyme Expression

To validate the protective effect of SFN on a mouse hepatocyte cell line AML12, the expression levels of two glutathione-S-transferase (GST) isoforms, P1 and A3 (denoted as GSTP1 and GSTA3, respectively) and NAD(P)H:quinone oxidoreductase (NQO1), were quantified using western blot analysis after incubating the AML12 cells with SFN for 12, 24, and 36 hours. Beta-actin was used as a loading control.

The effects of SFN (10 μM), MIL-88A MOF loaded with SFN (denoted as MIL-88A-SFN, 10 μM SFN final concentration), or a corresponding amount of unloaded MIL-88A MOF on the GST activity in AML12 cells were measured at 12, 24, or 48 hours after the treatment. The GST activity assay was performed in the presence of glutathione and 1-chloro-2,4-dinitrobenzene (a GST substrate). Measurements were performed in quadruplicate.

Mice bearing B16F1 tumor were intravenously administered with SFN or MIL-88A-SFN at a dose of 10 mg SFN per kg animal, through the tail vein in groups of eight. The expression levels of GSTP1 and GSTA3 in the liver and tumor tissues were measured by western blot at 24 and 48 h following the intravenous administration. Tissues from the groups of mice treated with PBS or MIL-88A were also analyzed.

Protective Effect of SFN-Loaded MIL-88A Against Doxorubicin

MTT assay was performed to quantify AML12 cell survival after 24-hour exposure to 10 μM doxorubicin (DOX). The cells were pretreated with SFN or with MIL-88A-SFN. Control cells include those incubated with DOX without pretreatment, those incubated with MIL-88A-SFN alone without incubation with DOX, and those incubated with phosphate buffered saline (PBS). The activities of caspases and CYP1A1/CYP1B1 of these cells were also measured.

Mice bearing a B16F1 tumor were intravenously administered through the tail vein in different groups of eight: (1) pretreatment with 25 mg of MIL-88A-SFN per kg of mouse at day 0 (corresponding to 3 mg SFN per kg of mouse according to the loading capacity of SFN in MIL-88A), followed by DOX at day 1 at 10 mg per kg of mouse; (2) pretreatment with SFN at 3 mg per kg of mouse at day 0, followed by DOX at day 1 at 10 mg per kg of mouse; (3) PBS injected at day 0, followed by DOX at day 1 at 10 mg per kg of mouse; and (4) PBS injected at both day 0 and day 1. Liver and blood samples were collected and analyzed to identify liver damage and morphology.

Results

Properties of SFN-Loaded MOF Particles

Three iron-based biocompatible MOFs (i.e., MIL-88A, MIL-100, MIL-88B_NH₂) with different crystalline structures, pore sizes/shapes and pore volumes were prepared, and their properties are shown in Table 1.

TABLE 1
Characteristics of SFN-loaded MOFs

| | | | | | Release | |
|---|---|---|---|---|---|---|
| | | | Particle | Pore | profile | Loading, |
| MOF | Formula | Linker [L] | size, Å | size, Å | (t_{1/2}), min | % wt |
| MIL-88A | Fe₃O(OH)(H₂O)[L]₃ | fumaric acid | 890 ± 85 | 5-7 | 15 | 12 ± 1 |
| MIL-100 | Fe₃O(OH)(H₂O)₂[L]₂ | trimesic | 104 ± 7 | 24-27 | 15 | 8 ± 1 |
| | | acid | | | | |
| MIL- | Fe₃O(OH)(H₂O)[L]₃ | amino- | 658 ± 80 | 7-9 | 60 | 17 ± 1 |
| 88B_NH₂ | | terephthalic | | | | |
| | | acid | | | | |

The efficacy of hepatoprotectant encapsulation into the pores of MOFs was finely tuned depending on the pore volume and the synthesis conditions of the chosen type of MOFs. Moreover, the hepatoprotectant release profile was modulated by the selection of MOF carriers.

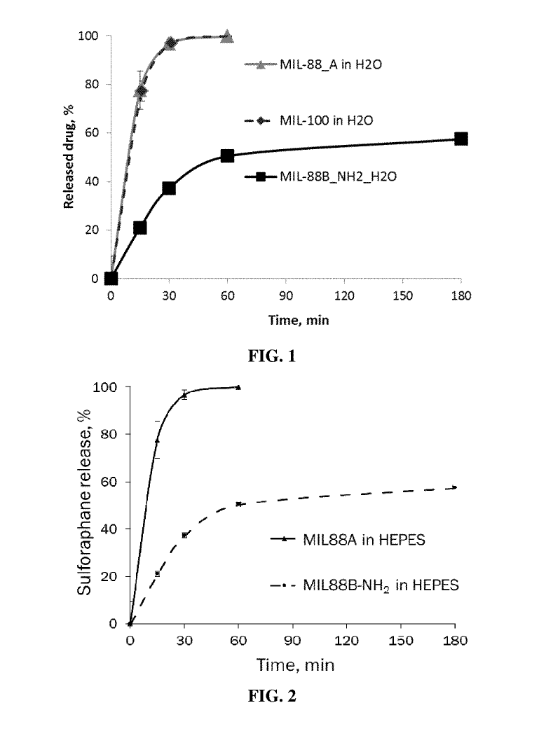

FIG. 1 shows that SFN was released faster from MIL-88A and MIL-100 than from MIL-88B_NH₂ in water. FIG. 2 shows that SFN was released faster from MIL-88A than from MIL-88B_NH₂ in HEPES buffer. Moreover, the release profile of SFN from MIL-88A in water is similar to its release profile in HEPES buffer; more than 90% of SFN was released from MIL-88A within 30 min in both water and HEPES buffer.

Protective Effect of SFN and SFN-Loaded MIL-88A

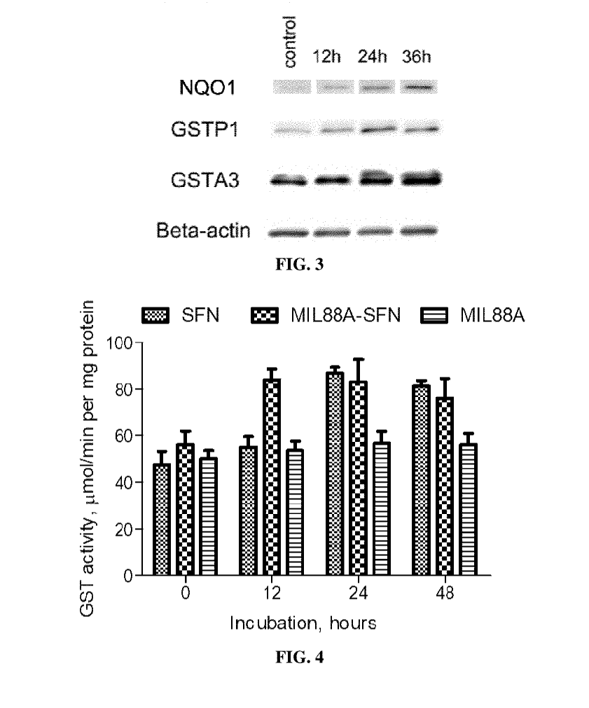

FIG. 3 shows that 24-h SFN treatment of AML12 cells led to upregulation of phase 2 enzymes, GSTP1 and GSTA3, compared to a control of untreated AML12 cells, as measured by western blot analysis. The SFN treatment also led to enhanced expression of NQO1, which regulates the redox potential in cells.

Next, the effects of treatment with SFN, MIL-88A, or SFN-loaded MIL-88A on the GST activity were measured. FIG. 4 shows that following 12 hours of incubation, the average GST activity in the cells treated with SFN-loaded MIL-88A was higher than the GST activity of the cells treated with SFN or with MIL-88A. At 24 and 48 hours, the GST activity in the cells treated with SFN equaled the GST activity in the cells treated with SFN-loaded MIL-88A. This result indicates that the MOF facilitated the uptake of SFN in the mouse hepatocytes.

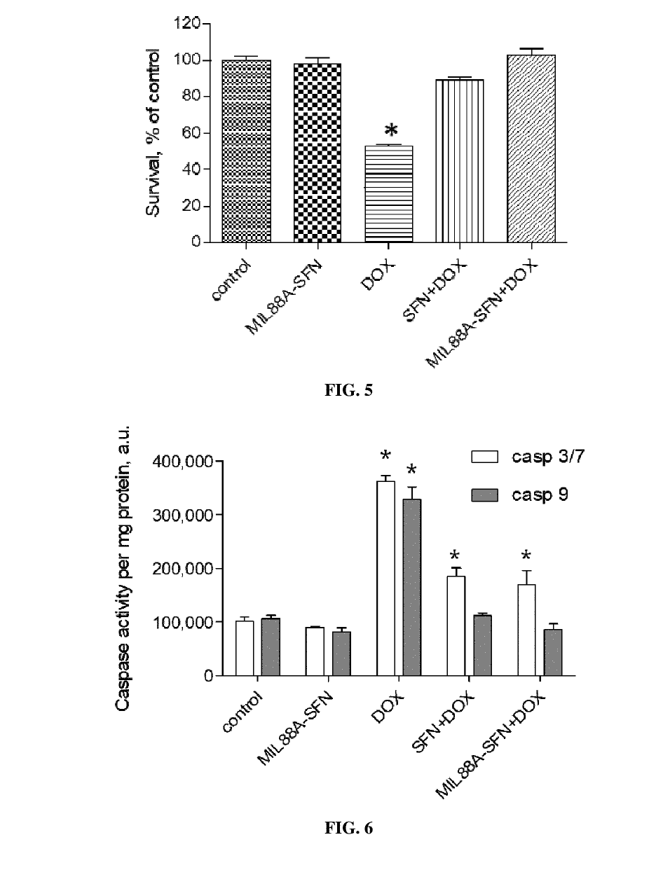

The effects of SFN and MIL-88A-SFN on AML12 cells treated with an anticancer drug, doxorubicin (DOX), were measured. FIGS. 5 and 6 show that pretreatment with SFN or MIL-88A-SFN significantly decreased apoptosis and the caspase activity. Cells treated with DOX alone had the lowest survival rate and highest caspase activity.

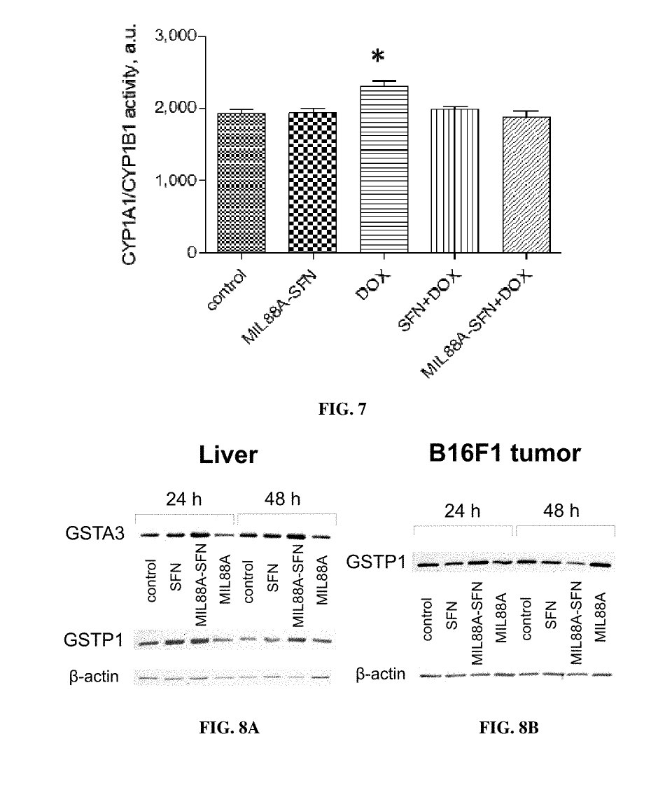

FIG. 7 shows that incubation with DOX induced an increase in the level of CYP1A1/CYP1B1. CYP1A1/CYP1B1 is involved in the generation of reactive oxygen species (ROS) and regulates oxidative stress in cells. Pretreatment with SFN or MIL-88A-SFN suppressed the upregulation of CYP1A1/CYP1B1 in the AML12 cells treated with DOX.

As shown in FIG. 8A, western blot analysis of the liver tissues of mice bearing B16F1 tumor shows that the MIL-88A-SFN treatment caused an upregulation of the expression levels of GSTs (i.e., GSTP1 and GSTA3) in the liver than SFN alone did (when SFN was dosed at 3 mg per kg of animal in both groups). Enhanced levels of GSTs in the liver persisted for a minimal of two days after the animal was administered with MIL-88A-SFN.

As shown in FIG. 8B, western blot analysis of the B16F1 tumor tissues of mice bearing B16F1 tumor shows that neither SFN nor MIL-88A-SFN strongly affected the GST expression in the B16F1 melanoma tumor tissues.

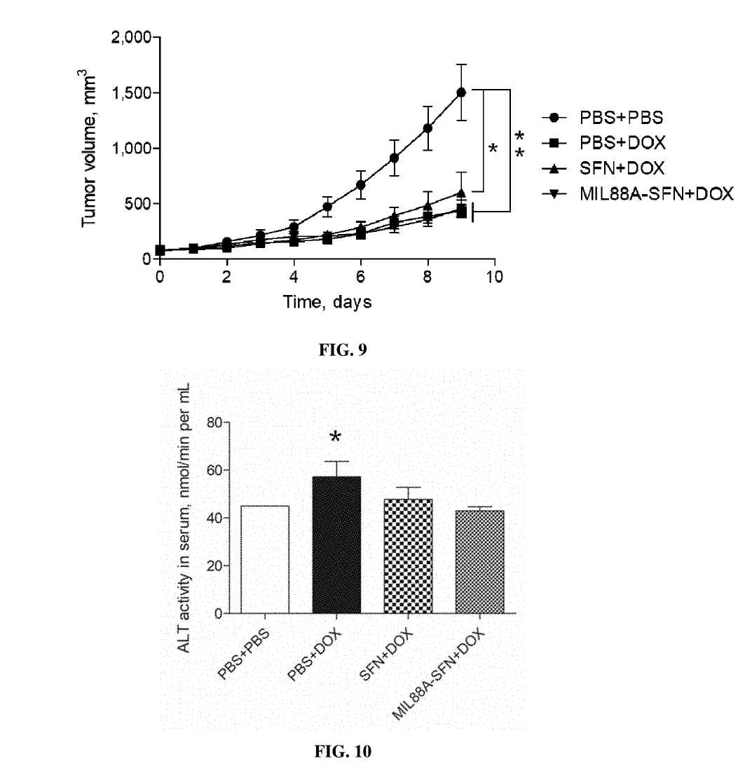

FIG. 9 shows that mice treated with DOX with or without pretreatment with either MIL-88A-SFN or SFN had lower tumor volumes up to at least 9 days compared to mice only receiving PBS at day 0 and day 1. Pre-treatment with MIL-88A-SFN did not affect the efficacy of DOX as a chemotherapeutic agent.

FIG. 10 shows that the DOX treatment induced an upregulation of the alanine aminotransferase (ALT) activity in blood serum of mice bearing B16F1 tumor, indicating drug-induced liver injury. Pre-treatment with SFN or MIL-88A-SFN attenuated the upregulation of the ALT activity.

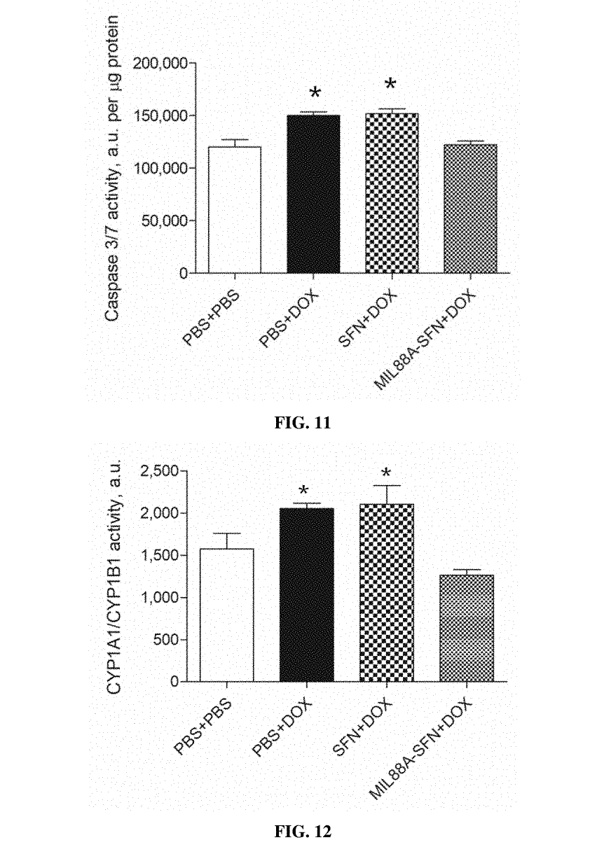

FIGS. 11 and 12 show that the DOX treatment induced an upregulation of the caspase activity and the CYP1A/CYP1B activity in the liver tissues of mice bearing B16F1 tumor. Pretreatment with MIL-88A-SFN, but not SFN, attenuated the upregulation, thereby inhibiting the DOX-induced pro-apoptotic processes and ROS-related damages in the liver.

Morphological microscopic analysis of the liver tissues determined that the DOX treatment induced moderate cholestasis, focal necrosis, and mononuclear infiltration of sinusoids. Pretreatment with SFN or MIL-88A-SFN attenuated the aforementioned symptoms of hepatotoxicity. Pretreatment with MIL-88A-SFN prevented cholestasis and almost completely prohibited other pathological changes induced by DOX.

### Example 2. PLGA Polymeric Particles Loaded with Silibinin (SBN) Mitigate Dacarbazine (DTIC)-Induced Liver Damage

Materials and Methods

Fabrication of PLGA Polymeric Particles Loaded with Silibinin

PLGA polymeric particles loaded with silibinin (SBN) were prepared by a single emulsion-solvent evaporation technique. Briefly, 40 mg PLGA (RESOMER® RG 502H, 50:50 copolymer of lactic acid and glycolic acid, molecular weight 7-17 kDa) and 4 or 12 mg of SBN were solubilized in 2 mL of acetone or in 1.9 mL acetone with addition of 100 μl DMSO, to achieve a 15 or 30% wt. loading rate, respectively. The prepared solution was homogenized for 5 min and slowly added to 2% PVA aqueous solution. The mixture was kept under stirring (500 rpm) overnight to evaporate the organic phase. The PLGA polymeric particles were collected by ultracentrifugation at 72,000×g for 15 min at 4° C. and washed three times with deionized water and collected again. The purified PLGA polymeric particles were lyophilized.

Measurement of SBN Loading Efficacy

Hepatoprotectant loading efficacy was determined using HPLC equipped with a UV-detector (Waters 1525). Chromatography was performed using a reversed-phase Luna C18 column (5 μm in particle size, 4.6 mm×150 mm in diameter). The mobile phase containing 50% v/v acetonitrile in 0.05% acetic acid aqueous solution was delivered at a flow rate of 0.5 mL/min. Chromatograms were monitored at 288 nm using an injected volume of 20 μL. The temperature of the column was set at 25° C. Standard solutions used for the calibration curve consisted of SBN dissolved in a 30:70 (v/v) tetrahydrofuran (THF)/acetonitrile mixture with concentrations of 500, 250, 125, 62.5, 31.25, 15.62, 7.81, 3.91 μg/mL. The calibration curve showed a good correlation coefficient, i.e., >0.99. The retention time of SBN was 9.1 min. In order to determine the loading efficacy of SBN, lyophilized PLGA particles containing SBN were suspended in the 30:70 (v/v) THF/acetonitrile mixture at a concentration of 0.5 mg/mL and filtered using 0.22 μm PVDF filters after a 2-hour incubation. The supernatant was assayed in HPLC measurements.

Measurement of SBN Release Profile

For release profile measurements, SBN-loaded PLGA particles were suspended in PBS at 37° C. 10% v/v of THF was added to fully dissolve released SBN. The suspension was filtered using 0.22 μm PVDF filters at different incubation times (from 30 min up to 7 days) and the supernatant was assayed using HPLC to determine the release amount at each collection time point.

Effect of SBN on Phase 2 Enzymes Expression

Western blot analysis was used to measure the expression levels of phase 2 enzymes, i.e., GSTP1 and GSTA3, in AML12 hepatocytes after 24 h and 48 h of exposure to 100 μM SBN.

For analysis of phase 2 enzyme expression in vivo, liver lysates were collected 24 h after injection of mice with either SBN or SBN-PLGA (10 mg SBN per kg of mouse). Liver lysates from cohorts of animals treated with plain PLGA particles or PBS were also collected. Western blot assay was used for analysis of GSTP1, GSTA3 and NQO1 expression. Beta-actin was used as a loading control.

Protective Effect of SBN-Loaded PLGA Particles

Mice bearing B16F1 tumor were intravenously administered through the tail vein in different groups of eight: (1) pretreatment with PLGA-SBN (corresponding to 10 mg SBN per kg of mouse), followed by dacarbazine (DTIC) on the next day at a dose of 120 mg per kg mouse; (2) pretreatment with SBN (10 mg per kg), followed by DTIC on the next day at a dose of 120 mg per kg mouse; (3) PBS, followed by DTIC on the next day at a dose of 120 mg per kg mouse; (4) pretreatment with plain PLGA and free (non-encapsulated) SBN (10 mg per kg of mouse), followed by PBS on the next day; and (5) PBS injected at both days (once per day).

Liver and blood samples were collected and analyzed to identify liver damage and morphology.

Results

Properties of SBN-Loaded PLGA Particles

The efficacy of SBN encapsulation into PLGA particles was tuned by altering synthesis conditions (i.e., the initial concentration of SBN), leading to two formulations with SBN occupying 15 and 30% total weight of the SBN-loaded PLGA particles.

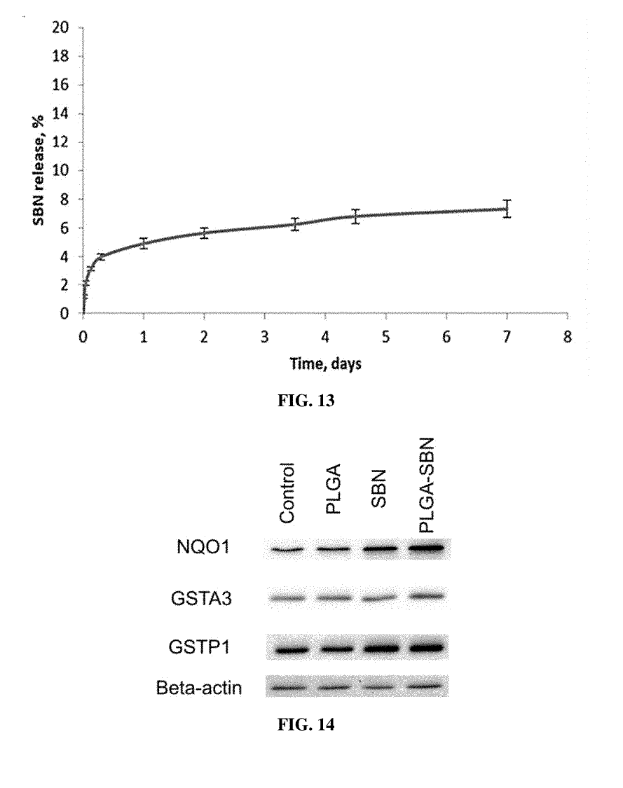

FIG. 13 shows that the SBN release rate from PLGA particles in PBS buffer is slow. After 7 days, about 8% SBN was released from the PLGA particles.

Protective Effect of SBN and SBN-Loaded PLGA Particles

Western blot analysis shows that exposure of AML12 hepatocytes to SBN enhanced expression of GSTP1 but not GSTA3. FIG. 14 shows that treatment of B16-F1 melanoma bearing C57BL/6J mice with free SBN or SBN-loaded PLGA particles (denoted as PLGA-SBN) led to pronounced upregulation of GSTP1 and NQO, but not GSTA3 in the liver tissues.

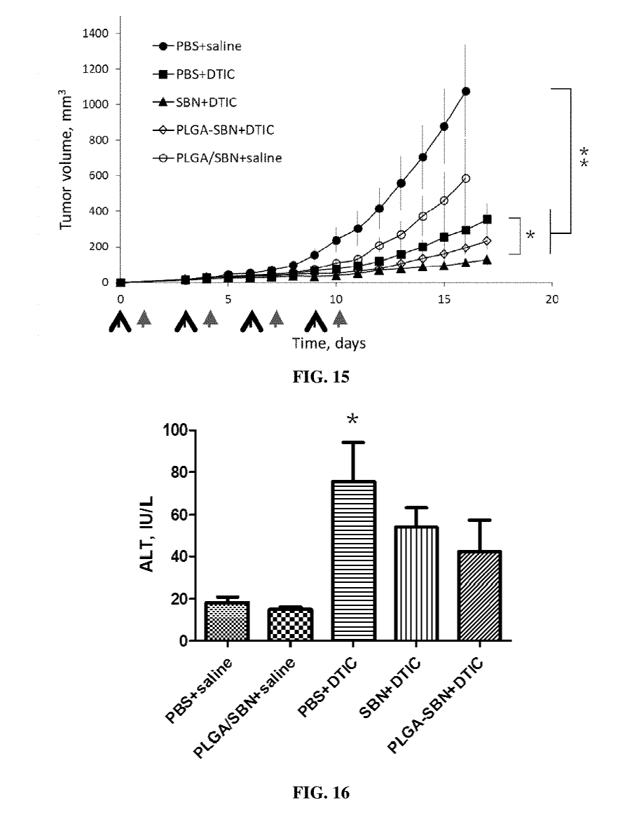

To determine whether pretreatment with SBN or PLGA-SBN affected the efficacy of chemotherapy with a chemotherapeutic agent, dacarbazine (DTIC), mice bearing B16F1 tumor were treated as mentioned in Methods. As shown in FIG. 15, pretreatment with SBN or PLGA-SBN particles did not affect the anti-cancer efficacy of DTIC.

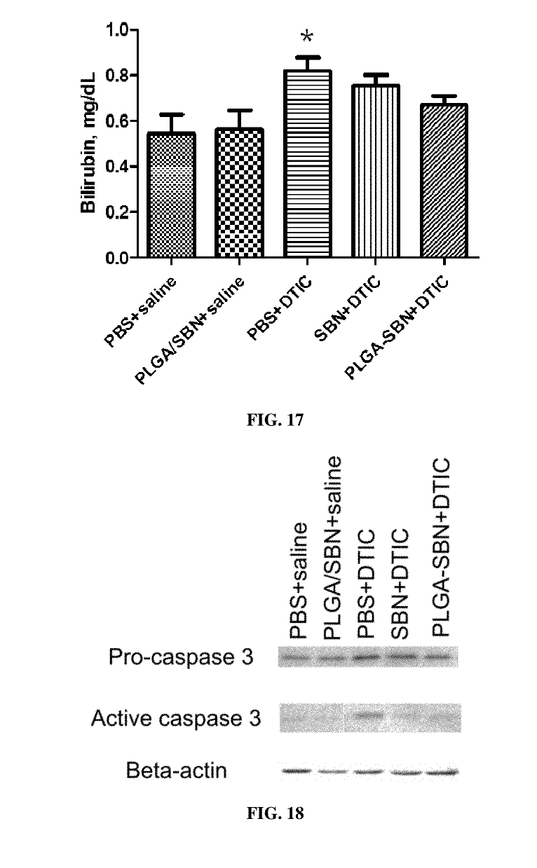

FIGS. 16 and 17 show that the DTIC treatment induced an upregulation of the alanine aminotransferase (ALT) activity and the bilirubin concentration in blood serum of mice bearing B16F1 tumor, indicating drug-induced liver injury. Pre-treatment with PLGA-SBN attenuated the upregulation of the ALT activity and the bilirubin concentration to a higher extent compared to pretreatment with unencapsulated SBN.

As shown in FIG. 18, western blot analysis demonstrated that the DTIC treatment induced caspase 3 activation in liver tissues of mice bearing B16F1 tumor. Pretreatment with SBN and PLGA-SBN attenuated the activation of caspase 3, thereby inhibiting the DTIC-induced pro-apoptotic processes in the liver.

Morphological analysis of liver tissues after chemotherapy with DTIC was performed. Mononuclear infiltration of sinusoids, being a prerequisite of veno-occlusive disease, is considered as the most dangerous hepatotoxic adverse effect induced by DTIC chemotherapy. PLGA-SBN particles effectively mitigated this pathological change as compared with the group with free SBN and the group without pretreatment. Moreover, pretreatment of PLGA-SBN also completely prevented DTIC-induced cholestasis and focal necrosis of hepatocytes.

Unless defined otherwise, all technical and scientific terms used herein have the same meanings as commonly understood by one of ordinary skill in the art to which the disclosed invention belongs. Publications cited herein and the materials for which they are cited are specifically incorporated by reference.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Those skilled in the art will recognize, or be able to ascertain using no more than routine experimentation, many equivalents to the specific embodiments of the invention described herein. Such equivalents are intended to be encompassed by the following claims.

We claim:

1. A system for chemotherapy comprising:
a first formulation, comprising short-circulating particles containing a hepatoprotectant, wherein the particles are nanoparticles, microparticles, or combinations thereof, and
a second formulation comprising a chemotherapeutic agent.

2. The system of claim 1, wherein the particles comprise a metal organic framework (MOF).

3. The system of claim 2, wherein the MOF comprises a metal ion selected from the group consisting of Fe³⁺, Fe²⁺, Zn²⁺, Na⁺, K⁺, Mg²⁺, Al³⁺, and Ca²⁺.

4. The system of claim 2, wherein the MOF comprises a linker selected from the group consisting of fumaric acid, trimesic acid, terephthalic acid, citric acid, malic acid, tartaric acid, methanoic acid, ethanoic acid, propanoic acid, butanoic acid, valeric acid, caproic acid, caprylic acid, capric acid, lauric acid, mylistic acid, palmitic acid, stearic acid, oleic acid, linoleic acid, linolenic acid, cyclohexanecarboxylic acid, phenylacetic acid, benzoic acid, oxalic acid, malonic acid, succinic acid, glutaric acid, adipic acid, pimelic acid, suberic acid, maleic acid, phthalic acid, isophthalic acid, hemimellitic acid, trimellitic acid, succinic anhydride, maleic anhydride, phthalic anhydride, glycolic acid, lactic acid, hydroxybutyric acid, mandelic acid, glyceric acid, gallic acid, malic acid, 3,3′,5,5′-azobenzene tetracarboxylic acid, zoledronic acid, tartaric acid, citric acid, ascorbic acid, imidazole, 1-methylimidazole, 2-methylimidazole, 4-methylimidazole, and γ-cyclodextrin, and derivatives thereof.

5. The system of claim 1, wherein the particles comprise a polymer selected from the group consisting of polyhydroxyesters, polycaprolactone, and polyanhydrides, and blends or copolymers thereof.

6. The system of claim 5, wherein the polymer is poly(lactic-co-glycolic acid) (PLGA).

7. The system of claim 1, wherein the hepatoprotectant is selected from the group consisting of coumarin, sulforaphane, erucin, berberine, dimethyl fumarate, silibinin, exemestane, amifostine, mesna, and dexrazoxane, and derivatives thereof.

8. The system of claim 1, wherein the first formulation comprises an effective amount of the hepatoprotectant to reduce liver toxicity caused by the chemotherapeutic agent in the second formulation, as measured by a downregulation of the levels of alanine aminotransferase and/or aspartate aminotransferase in blood serum, compared to when the second formulation is administered without the first formulation.

9. The system of claim 1, wherein the amount of the hepatoprotectant in the particles is within the range of 5 to 40% (w/w), 10 to 30% (w/w), or 10 to 20% (w/w).

10. Short-circulating particles containing a hepatoprotectant, wherein the particles are nanoparticles, microparticles, or combinations thereof.

11. The particles of claim 10, wherein the particles comprise a metal organic framework (MOF).

12. The particles of claim 11, wherein the MOF comprises a metal ion selected from the group consisting of Fe³⁺, Fe²⁺, Zn²⁺, Na⁺, K⁺, Mg²⁺, and Ca²⁺.

13. The particles of claim 11, wherein the MOF comprises a linker selected from the group consisting of fumaric acid, trimesic acid, terephthalic acid, citric acid, malic acid, tartaric acid, methanoic acid, ethanoic acid, propanoic acid, butanoic acid, valeric acid, caproic acid, caprylic acid, capric acid, lauric acid, mylistic acid, palmitic acid, stearic acid, oleic acid, linoleic acid, linolenic acid, cyclohexanecarboxylic acid, phenylacetic acid, benzoic acid, oxalic acid, malonic acid, succinic acid, glutaric acid, adipic acid, pimelic acid, suberic acid, maleic acid, phthalic acid, isophthalic acid, hemimellitic acid, trimellitic acid, succinic anhydride, maleic anhydride, phthalic anhydride, glycolic acid, lactic acid, hydroxybutyric acid, mandelic acid, glyceric acid, gallic acid, malic acid, 3,3′,5,5′-azobenzene tetracarboxylic acid, zoledronic acid, tartaric acid, citric acid, ascorbic acid, imidazole, 1-methylimidazole, 2-methylimidazole, 4-methylimidazole, and γ-cyclodextrin, and derivatives thereof.

14. The particles of claim 10, wherein the hepatoprotectant is selected from the group consisting of coumarin, sulforaphane, erucin, berberine, dimethyl fumarate, silibinin, exemestane, amifostine, mesna, and dexrazoxane, and derivatives thereof.

15. The particles of claim 10, wherein the amount of the hepatoprotectant in the particles is within the range of 5 to 40% (w/w), 10 to 30% (w/w), or 10 to 20% (w/w).

16. A method for administering a chemotherapeutic agent to a patient in need thereof using the system of claim 1, comprising:
(a) administering the first formulation and the second formulation to the patient.

17. The method of claim 16, wherein in step (a) the first formulation and the second formulation are administered simultaneously.

18. The method of claim 16, wherein the first formulation and the second formulation are administered sequentially, wherein the first formulation is administered prior to the second formulation.

19. The method of claim 16, further comprising after step (a):
(b) administering the second formulation one or more times.

20. The method of claim 16, wherein the first formulation provides controlled release of the hepatoprotectant for at least 1 day, 2 days, 3 days, 4 days, 5 days, 6 days, 7 days, or up to 1 month.
